(19) United States
(12) Patent Application Publication
(10) Pub. No.: US 2023/0043674 A1
(43) Pub. Date: Feb. 9, 2023

(54) SCIENTIFIC AND TECHNICAL SYSTEMS AND METHODS FOR PROVIDING HAIR HEALTH DIAGNOSIS, TREATMENT, AND STYLING RECOMMENDATIONS

(71) Applicant: TECHTURIZED, INC., Atlanta, GA (US)

(72) Inventors: Candace Mitchell, Atlanta, GA (US); Chanel Martin, Atlanta, GA (US); Robin D. Groover, Atlanta, GA (US); Stephen Pettus, Atlanta, GA (US); Kirubel Girma, Atlanta, GA (US)

(21) Appl. No.: 17/397,864

(22) Filed: Aug. 9, 2021

(51) Int. Cl. G16H 50/30; G16H 30/40; G16H 20/00

(52) U.S. Cl. CPC G16H 50/30; G16H 30/40; G16H 20/00

(57) ABSTRACT

Disclosed herein is a hair health assessment, treatment, and styling diagnosis and recommendation method and system. The techniques use science and technology in order to aid users in achieving optimal health and beauty of their hair. The present disclosure allows a user to provide hair samples, such as digital images of hair, that can be analyzed using Machine Learning (ML) and Artificial Intelligence (AI) technology to provide recommendations including hair health assessments, hair care regimes, hair care products, and hair care services in an accurate, efficient, and automated manner. The present disclosure may employ a digital image capture device, either stand alone device or an embedded camera of a mobile device or a microscopic attachment, in order to capture digital images of their hair at a smartphone camera magnification level from a selfie image or microscopic-level of magnification, as a hair sample to be analyzed by the system.

### FIELD OF DISCLOSURE

The present disclosure generally relates to software and hardware technology for assessment and preparation affecting hair health, growth, and care of the hair and/or skin.

### BACKGROUND OF THE DISCLOSURE

In some situations, users may desire personalized assessments for hair care. For example, a user may try countless hair stylists and hair care products, and watch many different hair tutorials. Thus, the conventional strategy is a process of trial and error. This often causes problems because the conventional strategy does not account for the users unique hair characteristics, and oftentimes requires costly visits to a professional hair stylist, a medical professional, or resorts to applying chemical hair treatments.

Accordingly, there remains a need for improved methods for providing personalized hair care assessments and recommendations. This need and other needs are satisfied by the various aspects of the present disclosure.

### BRIEF OVERVIEW

Scientific systems and methods for providing hair health assessment and styling recommendations may be provided. For example, a system including a hair health assessment diagnosis and recommendation platform can be provided. Further, the present disclosure can use science and technology in order to aid women in achieving their optimal health and beauty of their hair.

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

The present disclosure may use science and technology in order to aid women in achieving their optimal health and beauty of their hair. This optimum hair health may be termed hair nirvana. The present disclosure may allow customers to receive hair assessment recommendations based on the disclosed Scientific and Technical Systems and Methods for Providing Hair Health Assessment and Styling Recommendations.

In one aspect, the present disclosure may provide for a method for sending hair strand samples (also referred to herein as specimens) from one or more regions on the customer's head. In another aspect, the present disclosure may provide for a method of sending digital hair images of hair strand samples from one or more regions on the customer's head. According to the embodiments, the digital hair images may be captured using an image capture device, for instance an embedded digital camera of a mobile device (e.g., front-facing digital camera of a smartphone) or a digital microscope that may be used as a separate device or employed as an attachment to a mobile device.

In further aspects, the present disclosure may provide a scanning electron microscope (SEM), digital USB microscope, or imaging device including but not limited to a Hitachi S 3700N Variable Pressure SEM or a MicroSight 5MP, to analyze these one or more hair samples. In another aspect, the present disclosure may provide for a microscope apparatus configured to attach to a mobile device.

With an SEM or microscopic imaging device, the present disclosure allows for determining the condition and current health of one or more customers' hair strands from the hair samples. In another aspect, the present disclosure allows for scientific analysis of the one or more hair strands from the one or more hair samples. In yet another aspect, the present disclosure may provide a user interface wherein one or more customers may submit additional data associated with the one or more hair samples.

In further aspects, the present disclosure may provide for the determining of one or more characteristics of the one or more hair samples. In yet another aspect, the present disclosure may provide for the determining of one or more characteristics of the one or more hair samples based on the analysis of the one or more hair samples and/or submitted data. In other aspects, the present disclosure may provide for classification of the one or more hair samples based on a determination of one or more characteristics.

In further aspects, the present disclosure may be configured such that all information and data gathered may allow the disclosure to provide one or more recommendations. In further aspects, the present disclosure may provide recommendations which may include but not be limited to hair care regimes, hair assessments, hair care products, hair ingredients, and other solutions that may be necessary for one or more customers to promote or maintain healthy beautiful hair. In yet even other aspects, the present disclosure may assist one or more customers in achieving one or more desired hair styles based on one or more methods or assessments. In further aspects, the present disclosure may provide additional recommendations that will fit one or more customer's specific lifestyle and/or living environment, and personal preferences.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

In accordance with the purposes of the disclosure, as embodied and broadly described herein, the disclosure, in one aspect, relates to Scientific and Technical Systems and Methods for Providing Hair Health Assessment and Styling Recommendations, such as, for example recommending hair care products and personalized hair care plans based on data and scientific analysis of hair specimens. In further aspects, the present disclosure may provide real-time data gathering systems and methods. In yet another aspect, the present disclosure may provide systems and methods for delivering real-time recommendations and personalized hair care plans to customers.

In another aspect, the disclosure relates to methods and systems for providing methods and systems for electronic hair records documentation.

In further aspects, the disclosure also relates to skin analysis, health analysis, wardrobe analysis, face analysis, or other types of analysis. In yet even further aspects, the present disclosure relates to providing user recommendations and/or personalized hair care plans for skin care, health care, cosmetics, wardrobe, fashion, and/or other type of customized user recommendations. In other aspects, the disclosure may provide systems and methods for providing customized user recommendations relating to but not limited to events, lifestyle, makeup, cosmetics, fashion, skincare, health and wellness, food, and the like.

Additional aspects of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are explanatory only and are not restrictive of the disclosure, as claimed.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicants. The Applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1A illustrates a block diagram of an operating environment consistent with the embodiments disclosed herein.

FIG. 1B illustrates a block diagram of an operating environment consistent with the embodiments disclosed herein.

FIG. 1C illustrates a block diagram of an operating environment consistent with the embodiments disclosed herein.

FIG. 1D illustrates a block diagram of an example technology stack implementing the various functions and features consistent with the embodiments disclosed herein.

FIG. 2 illustrates a business user interface module consistent with the embodiments disclosed herein.

FIG. 3A shows a depiction of an application programing interface (API) and operating systems in accordance with an embodiment of the present disclosure.

FIG. 3B shows a depiction of a personalized hair care plan system in accordance with an embodiment of the present disclosure.

FIG. 3C shows a depiction of a hair care system in accordance with an embodiment of the present disclosure.

FIG. 3D shows a depiction of a hair care software application in accordance with an embodiment of the present disclosure.

FIG. 4 shows a depiction of an operating system infrastructure in accordance with an embodiment of the present disclosure.

FIG. 5A and FIG. 5B show a flow chart of a method for providing Scientific and Technical Systems and Methods for Providing Hair Health Assessment and Styling Recommendations.

FIG. 6A shows a depiction of hair care software application embodiment for providing Scientific and Technical Systems and Methods for Providing Hair Health Assessment and Styling Recommendations in accordance with an embodiment of the present disclosure.

FIG. 6B shows a depiction of a method for providing Scientific and Technical Systems and Methods for Providing Hair Health Assessment and Styling Recommendations in accordance with an embodiment of the present disclosure.

FIG. 6C shows a depiction of a method for providing Scientific and Technical Systems and Methods for Providing Hair Health Assessment and Styling Recommendations in accordance with an embodiment of the present disclosure.

FIG. 6D shows a depiction of a hair care software application embodiment for providing Scientific and Technical Systems and Methods for Providing Hair Health Assessment and Styling Recommendations in accordance with an embodiment of the present disclosure.

FIG. 6E shows a depiction of a hair care software application embodiment for providing Scientific and Technical Systems and Methods for Providing Hair Health Assessment and Styling Recommendations in accordance with an embodiment of the present disclosure.

FIG. 7 is a block diagram of a system including a computing device for performing the various functions and methods disclosed herein.

FIG. 8 shows a depiction of a graphical user interfaces (GUIs) that may be presented by the hair care software application embodiment for providing Scientific and Technical Systems and Methods for Providing Hair Health Assessment and Styling Recommendations in accordance with an embodiment of the present disclosure.

FIG. 9 shows a depiction of a graphical user interfaces (GUIs) that may be presented in accordance with an embodiment of the present disclosure.

FIG. 10 shows a depiction of a graphical user interfaces (GUIs) that may be presented in accordance with an embodiment of the present disclosure.

FIG. 11 shows a depiction of a graphical user interfaces (GUIs) that may be presented in accordance with an embodiment of the present disclosure.

FIG. 12 shows a depiction of a graphical user interfaces (GUIs) that may be presented in accordance with an embodiment of the present disclosure.

FIG. 13 shows a depiction of a graphical user interfaces (GUIs) that may be presented in accordance with an embodiment of the present disclosure.

FIG. 14 shows a depiction of a graphical user interfaces (GUIs) that may be presented in accordance with an embodiment of the present disclosure.

FIG. 15 shows a depiction of a graphical user interfaces (GUIs) that may be presented in accordance with an embodiment of the present disclosure.

FIG. 16 shows a depiction of a graphical user interfaces (GUIs) that may be presented in accordance with an embodiment of the present disclosure.

FIG. 17 shows a depiction of a graphical user interfaces (GUIs) that may be presented in accordance with an embodiment of the present disclosure.

FIG. 18 shows a depiction of a graphical user interfaces (GUIs) that may be presented in accordance with an embodiment of the present disclosure.

FIG. 19 shows a depiction of a graphical user interfaces (GUIs) that may be presented in accordance with an embodiment of the present disclosure.

FIG. 20 shows a depiction of a graphical user interfaces (GUIs) that may be presented in accordance with an embodiment of the present disclosure.

FIG. 21 shows a depiction of a graphical user interfaces (GUIs) that may be presented in accordance with an embodiment of the present disclosure.

FIG. 22 shows a depiction of a graphical user interfaces (GUIs) that may be presented in accordance with an embodiment of the present disclosure.

FIG. 23 shows a depiction of a graphical user interfaces (GUIs) that may be presented in accordance with an embodiment of the present disclosure.

FIG. 24 shows a depiction of a graphical user interfaces (GUIs) that may be presented in accordance with an embodiment of the present disclosure.

FIG. 25 shows a depiction of a graphical user interfaces (GUIs) that may be presented in accordance with an embodiment of the present disclosure.

FIG. 26 shows a depiction of a graphical user interfaces (GUIs) that may be presented in accordance with an embodiment of the present disclosure.

FIG. 27 shows a depiction of a graphical user interfaces (GUIs) that may be presented in accordance with an embodiment of the present disclosure.

### DETAILED DESCRIPTION OF THE DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of scientific and technical systems and methods for providing hair health assessment and styling recommendations, embodiments of the present disclosure are not limited to use only in this context. The present disclosure can be understood more readily by reference to the following detailed description of the disclosure and the Examples included therein.

Before the present articles, systems, devices, and/or methods are disclosed and described, it is to be understood that they are not limited to specific manufacturing methods unless otherwise specified, or to particular materials unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

All publications mentioned herein are incorporated herein by reference to disclose and describe the methods and/or materials in connection with which the publications are cited.

### a. Definitions

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used in the specification and the appended claims, the singular forms “a,” “an” and “the” include plural referents unless the context clearly dictates otherwise. Thus, for example, reference to “an opening” can include two or more openings.

Ranges can be expressed herein as from one particular value, and/or to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Disclosed are the components to be used to manufacture the disclosed devices, systems, and articles of the disclosure as well as the devices themselves to be used within the methods disclosed herein. These and other materials are disclosed herein, and it is understood that when combinations, subsets, interactions, groups, etc. of these materials are disclosed that while specific reference of each various individual and collective combinations and permutation of these materials cannot be explicitly disclosed, each is specifically contemplated and described herein. For example, if a particular material is disclosed and discussed and a number of modifications that can be made to the materials are discussed, specifically contemplated is each and every combination and permutation of the material and the modifications that are possible unless specifically indicated to the contrary. Thus, if a class of materials A, B, and C are disclosed as well as a class of materials D, E, and F and an example of a combination material, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the articles and devices of the disclosure. Thus, if there are a variety of additional steps that can be performed it is understood that each of these additional steps can be performed with any specific aspect or combination of aspects of the methods of the disclosure.

It is understood that the devices and systems disclosed herein have certain functions. Disclosed herein are certain structural requirements for performing the disclosed functions, and it is understood that there are a variety of structures that can perform the same function that are related to the disclosed structures, and that these structures will typically achieve the same result.

### I. Platform Overview

Consistent with embodiments of the present disclosure, a scientific and technical systems and methods for providing hair health assessment and styling recommendations may be provided. This overview is provided to introduce a selection of concepts in a simplified form that are further described below. This overview is not intended to identify key features or essential features of the claimed subject matter. Nor is this overview intended to be used to limit the claimed subject matter's scope. The Scientific and Technical Systems and Methods for Providing Hair Health Assessment and Styling Recommendations may be used by individuals or companies to provide customized user personalized hair care plans based on user data and scientific analysis.

The present disclosure provides a hair assessment diagnosis and recommendation platform. The present disclosure may use science and technology in order to aid women in achieving their optimal health and beauty of their hair. The present disclosure allows customers to electronically receive hair assessment recommendations based on the disclosed scientific and technical systems and methods for providing hair health assessment and styling recommendations, for example recommendations for hair products, ingredients, regimens, techniques, and professional services can be automatically added to an online profile for a user and stored in an electronic hair record.

In one aspect, the present disclosure provides for a method for sending hair samples from one or more regions on the customers head. In another aspect, the present disclosure provides for a method of sending digital hair images of hair samples from one or more regions on the customers head. In further aspects, the present disclosure provides a scanning electron microscope (SEM), digital USB microscope, or imaging device including but not limited to a Hitachi S 3700N Variable Pressure SEM or MicroSight 5MP, to analyze these one or more hair samples. In another aspect, the present disclosure provides for a microscope apparatus configured to attach to a mobile device. Accordingly, a mobile device, such as a smartphone, can be used to obtain highly magnified images of a user's hair strands to submit to the system for further analysis.

In another aspect, the physical hair samples may be received and analyzed by a medical professional, hair specialist, or technician in a salon, store, laboratory or science center. In yet another aspect the one or more hair images, hair data, image data, and user data may be received and analyzed by a device in a kiosk, medical professional, hair specialist, or technician in a laboratory or science center. In further aspects, the one or more hair samples, hair images, hair data, image data, and user data may be received and analyzed by an automated system, a computing system, or a program based on data compiled, studies conducted, or research from libraries, medical professionals, hair specialists, or technicians.

With an SEM or imaging device, the present disclosure allows for determining the condition and current health of one or more customers' hair strands from the hair samples. In another aspect, the present disclosure allows for scientific analysis of the one or more hair strands from the one or more hair samples. In yet another aspect, the present disclosure provides a user interface wherein one or more customers may submit additional data associated with the one or more hair samples.

In further aspects, the present disclosure provides for the determining of one or more characteristics of the one or more hair samples. In yet another aspect, the present disclosure provides for the determining of one or more characteristics of the one or more hair samples based on the analysis of the one or more hair samples and/or submitted data. In other aspects, the present disclosure provides for classification of the one or more hair samples based on a determination of one or more characteristics.

In further aspects, the present disclosure may be configured such that all information and data gathered may allow the disclosure to provide one or more recommendations. In further aspects, the present disclosure provides recommendations which may include but not be limited to hair care regimes, hair assessments, hair care products, and other solutions that may be necessary for one or more customers to promote or maintain healthy beautiful hair. In yet even other aspects, the present disclosure may assist one or more customers in achieving one or more desired hair styles based on one or more methods or assessments. In further aspects, the present disclosure provides additional recommendations that will fit one or more customer's specific lifestyle and/or living environment.

In accordance with the purposes of the disclosure, as embodied and broadly described herein, the disclosure, in one aspect, relates to Scientific and Technical Systems and Methods for Providing Hair Health Assessment and Styling Recommendations, such as, for example recommending hair care products and personalized hair care plans based on data and scientific analysis of hair specimens. In further aspects, the present disclosure provides real-time data gathering systems and methods. In yet another aspect, the present disclosure provides systems and methods for delivering real-time recommendations and personalized hair care plans to customers.

In another aspect, the disclosure relates to methods and systems for providing methods and systems for electronic hair records documentation.

In further aspects, the disclosure also relates to skin analysis, wardrobe analysis, or other types of analysis. In yet even further aspects, the present disclosure relates to providing user recommendations and/or a personalized care plans for hair care, skin care, health care, cosmetics, wardrobe, fashion, and/or other type of customized user recommendations in any industry. In other aspects, the disclosure provides systems and methods for providing customized user recommendations relating to but not limited to events, lifestyle, makeup, cosmetics, fashion, skincare, health and wellness, food, and the like.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

While aspects of the present disclosure may be limited to hair diagnosis and recommendation methods, systems and platforms, one of skill in the art will understand that each aspect of the present disclosure can be described and claimed in any statutory class.

The present disclosure may be implemented and embodied as a software application, a hardware device, a combination of both software and hardware, or an integration within existing software and/or websites. Each and every aspect of the present disclosure may be implemented as a software application, hardware device, combination of both software and hardware, or integration within existing software including but not limited to: an image capture system; a mobile application, a cloud computing server, an artificial intelligence module, an interactive questionnaire; a user interface module configured for user feedback; a recommendation engine configured to recommend products, ingredients, personalized hair care plans, and regimens; a microscopic image analysis module; a machine learning module configured to automate the process.

In one or more aspects, the present disclosure may provide a system configured such that hair strands may be manually collected and sent to a lab to be examined under a microscope. In other aspects, the present disclosure may provide a system that may be adapted for scalability and to allow for increased operational capacity by processing hair strand images instead of processing hair strands manually.

In one aspect, the present disclosure may take a microscopic image of hair captured by a smartphone camera apparatus, such as an embedded digital camera of a mobile device (or smartphone) and/or a microscope attachment (e.g., digital USB microscope), in order to detect a user's hair texture, type, and condition to recommend hair care regimens. The present disclosure may be further adapted to recommend additional information based on a user's profile including products, ingredients, regimens, techniques, and professional services. The present disclosure may be adapted to more efficiently solve the problem of users identifying their hair type, discovering the right hair products, and determining which products will provide the best results, while also requiring the proper hair care regimen. In one or more aspects, images may be captured through a hardware device with a microscopic camera then delivered to a cloud server 110 for analysis. Hardware devices may be situated throughout the hair industry ecosystem, primarily in hair salons and beauty supply stores.

In one or more aspects, the present disclosure may be implemented or embodied as a smartphone or mobile device having a microscopic image capture attachment. In further aspects, the present disclosure may provide the ability to automate a process requiring processing of manually collected hair strands. In yet another aspect, the present disclosure may provide for the use of a high-powered scanning electron microscope (SEM) or digital USB microscope. In yet even further aspects, the present disclosure may provide for machine learning modules configured to provide automatic hair strand detection to categorize and recommend assessments and products. In further aspects, the present disclosure may provide for artificial intelligence modules configured such that hair product recommendations may be provided instantly in real-time based on the processed hair strand information.

In one or more aspects, the present disclosure may also provide social sharing with a user community such as taking selfie photos to share updates on their hair journey. In other aspects, the present disclosure may be adapted to include a user taking a quiz or questionnaire. In certain embodiments, the quiz or questionnaire may not be included.

In one or more aspects, the present disclosure may provide a mobile device which may be adapted to provide a smartphone microscope configured to enable a user's mobile device to be transformed into a microscope. In yet another aspect, the present disclosure may be adapted to capture photos at the magnification level required to accurately analyze and detect the current state of a hair strand. In one or more embodiments, the mobile device or smartphone may need to be configured such that the mobile device may need to be configured at a predetermined distance from the hair specimen to accurately take an image. For example, the smartphone may be predetermined to capture an image within 2 inches to take an accurate picture.

In one or more aspects, the present disclosure may be configured such that a mobile application may enable specimen photos to be uploaded to a server. Specimen photos may include but is not limited to a front-facing image, angled image, or side profile image, and images on all sides of the head wherein the image comprises images from top left, top right, bottom left, bottom right, crown, and detailed microscopic images of one or more hair strands. In other aspects, the present disclosure may provide a cloud server configured such that cloud computing may enable the specimen photos to be stored and analyzed. In yet other aspects, the present disclosure may provide for machine learning modules which may be configured such that it may enable the photos to be translated into data, automatically detect the hair strand attributes, and instantly categorize or classify the information.

In one or more aspects, the present disclosure may be configured to provide a software recommendation engine further configured to enable the hair strand data and user submitted questionnaire data to return an accurate result. In another aspect, the present disclosure may be configured to provide an artificial intelligence module further configured to allow a recommendation engine to improve efficiency and accuracy upon subsequent analysis as a manner of getting smarter and smarter as more images may be uploaded, more products may be added to the system, more hair combinations may be matched to products, more ingredients, regimens, techniques, professional services recommendations and other elements. In yet other aspects, the present disclosure may provide the customer data, recommendations, product information, results, and other information may be stored for future uses including retail marketing, improving market research, further improving product fulfillment, and integrating with various parts of the hair industry ecosystem among other uses. In yet even further aspects, the present disclosure may provide user data such that it may be sold or distributed to manufacturers, salons, stylists, companies, businesses, and others.

In one or more instances, the present disclosure may be implemented using a skin care and/or hair care analysis device having a USB handheld microscope. In yet another aspect, the present disclosure may be implemented using a USB microscope, computer, and manufacturer software further adapted for specific use with the present disclosure. In yet another aspect, the present disclosure may be implemented using an integrated software and hardware device configured to send data to the cloud for analysis. In yet another aspect, the present disclosure may provide for a device, platform, method and system which may further incorporate voice data and/or voice command functionality configured to allow users to interact with the platform verbally. The present disclosure may be further configured to update the software on new hair needs or product needs and return new results.

In one or more instances, the present disclosure may provide for a mobile application which may be changed to provide for uploading photos, emailing the photos to the server, submitting the photos through an online form, sending photos in the mail, or other communication method (i.e. audio signal, Bluetooth, infrared, wi-fi, telecommunications signal and the like). In one or more instances, the present disclosure may provide for storage options including but not limited to cloud computing, local storage, blockchain storage, cold storage, flash drive storage, solid state storage devices, storage area networks, trusted network storage, encrypted storage devices, and the like. In one or more instances, the present disclosure may provide for machine learning modules and methods of categorizing images manually. In one or more instances, the present disclosure may provide a software recommendation engine and manual data collection including but not limited to using a spreadsheet to filter products based on certain data attributes.

In one or more instances, the present disclosure may provide for automating the analysis based on a hair image provided by a user. In yet another instance instances, the present disclosure may provide for a competitive advantage. In further aspects, the present disclosure may provide for updating and providing personalized guidance from user input on demand. In still further aspects, the present disclosure may provide for real-time data gathering processes from one or more customers. In yet further aspects, the present disclosure may provide for delivering real-time recommendations and personalized hair care plans to customers. In one or more instances, the present disclosure may provide specifically hair care recommendations. In one or more other instances, the present disclosure may provide for more expansive uses including but not limited to: skin analysis, health analysis, face analysis, wardrobe analysis, or other types of analysis.

In further aspects, the present disclosure may provide for future uses and contexts of including but not limited to event recommendations, event notifications, lifestyle recommendations, makeup recommendations, cosmetics recommendations, fashion recommendations, skincare recommendations, health and wellness recommendations, food recommendations, and other customized recommendations.

In further aspects, the present disclosure may provide for Electronic Hair Records documenting and management. In yet other aspects, the present disclosure may provide for electronic records management including but not limited to: medical, event, lifestyle, makeup, cosmetics, fashion, skincare, health and wellness, food, and other customized records. In yet further aspects, the platform may be embodied as a device wherein the device may further be embodied as a kiosk. In other aspects, the kiosk may be located at a store, salon, or other conceivable location.

### II. Platform Configuration

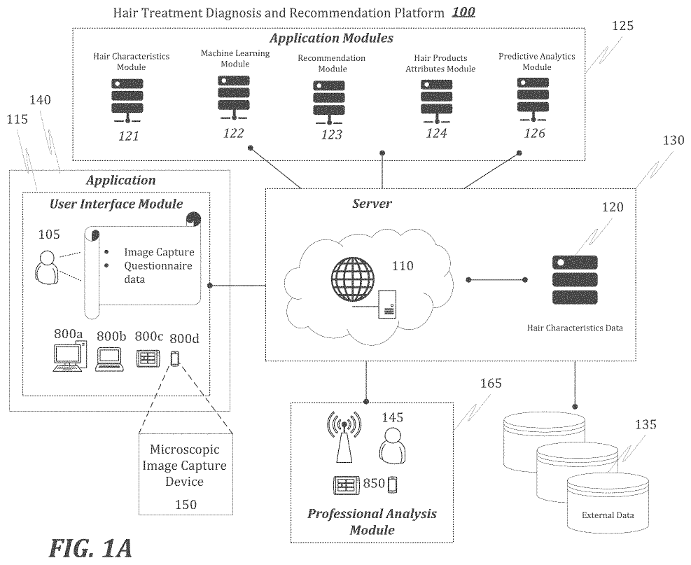

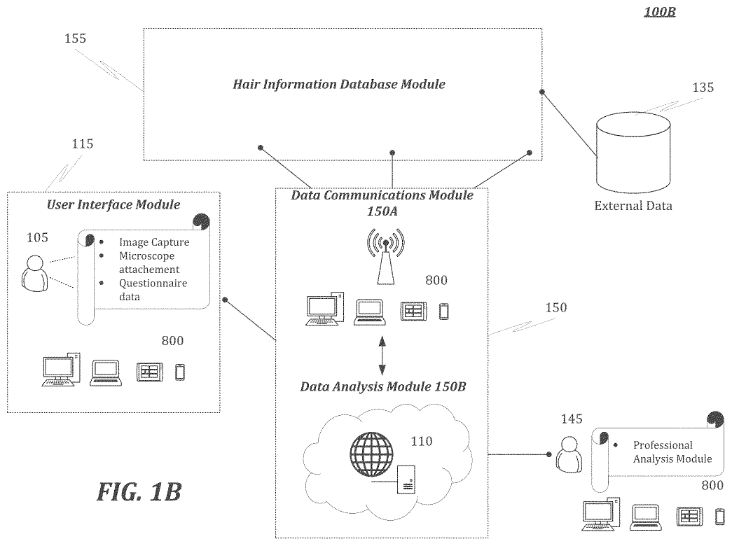

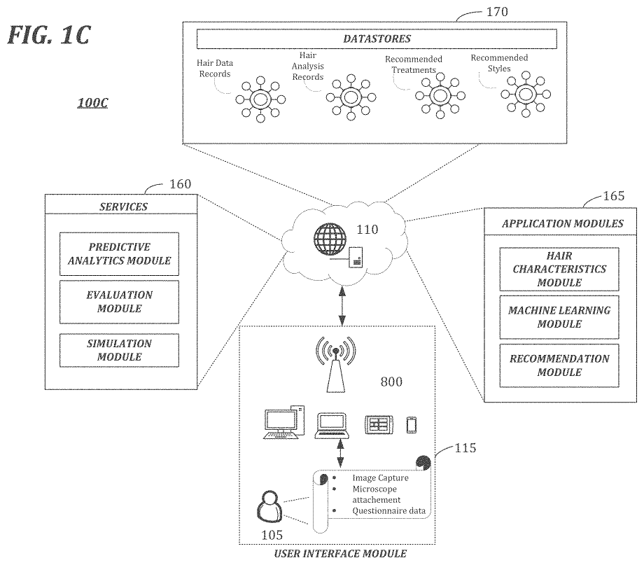

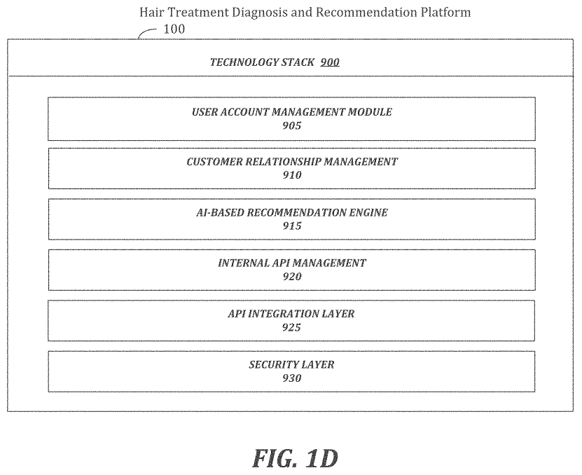

FIG. 1A illustrates an example of an operating environment through which a platform consistent with embodiments of the present disclosure may be provided. As referred to herein, a platform can generally refer to a computer-based system that includes a plurality of hardware components (e.g., computer devices, servers, programmable integrated circuits, etc.), software components (e.g., software applications, mobile applications (apps), graphical user interfaces (GUIs), plug-ins, computer software extensions, etc.), and any combination thereof. By way of a non-limiting example, a hair assessment diagnosis and recommendation platform 100 may be hosted on a centralized server 110, such as, a cloud computing service. The platform 100 allows one or more users, shown as a user 105, to receive customized hair product recommendations intelligently using a combination of professional hair experts and AI/ML technology that is primarily based on image segmentation and classification.

A user 105 may access the platform 100 through a software application 140. The software application 140 may be implemented by machine-readable instructions stored on a memory and executed by one or more processor(s) of the computing devices 800a-800d. The software application 140 may be embodied as, for example, but not be limited to, a website, a web application, a desktop application, a tablet application, and a mobile application, where each form of the software application 140 is appropriately compatible with a respective one of the computing devices 800a-800d. In the example, the computing devices are shown as a desktop computer 800a, a laptop computer 800b, a table computer 800c, and a smartphone 800d.

One possible embodiment of the software application 140 may be provided by the Myavana™ suite of products and services provided by Techturized™ Inc. In some embodiments, the software application 140 can be included with any necessary hardware (e.g., digital microscope) as component of a hair analysis kit. In some cases, the software application 140 may be implemented using subscription-based access for the user 105, which includes web-based access to the automated hair care recommendations that are generated by the platform's 100 Machine Learning (ML) and Artificial Intelligence (AI) technology. Moreover, the software application 140 can include software plug-ins or web browser extensions that, which serves as an extension to the functions of the host software application 140. The plug-ins or extensions can be employed to integrate the platform's 100 capabilities across the systems of different consumers, retailers, end users (e.g., via websites). For example, a retailer, such as Target™ may have a Myavana™ plug-in on their website, which accesses the automated hair care functions of the platform 100 in order to provide personalized recommendations to online shoppers. A user may have a Myavana™ browser extension installed on a web browser like Google Chrome or Safari, which accesses the automated hair care functions of the platform 100 in order to provide personalized recommendations to online shoppers. Even further, in some cases, the application software 140 can support Application Program Interface (API) access for receiving and/or distributing related data to hair product manufacturers and retailers. In general, the software application 140 (and other aspects of the platform 100) is designed for integration across the entire hair care industry ecosystem in a manner that allows a plurality of retailers, salons, hair care professionals, consumers, and end users to easily access and employ.

According to the embodiments, the software application 140 is particularly configured to obtain one or more microscopic digital images of a user's 105 hair. The digital images of hair can be captured by stand-alone digital microscopes, as well as a smartphone digital cameras, also referred to herein as an image capture device 150. Referring to the example of FIG. 1A, the smartphone 800d has the image capture device 150 that can be implemented as an attachment to a digital camera of a smartphone 800d or alternatively can be integrated thereon. For example, the image capture device 150 can be a microscopic lens having microscopic-level magnification, that is attached to the front-facing lens of the smartphone's 800d digital camera. Accordingly, the image capture device 150 particularly enables the smartphone 800d to digitally capture microscopic digital images of hair, for instance a strand of hair at the microscopic level. As a general description, the image capture device 150 is designed to function similar to a digital microscope that is functionally and physically integrated within the smartphone 800d. Thus, the image capture device 150 enables the user's smartphone 800d to be employed as a digital microscope to capture digital images of hair at the magnification level required to accurately analyze and detect the current state of the hair strand(s) of user 105. In an alternate embodiment, the image capture device 150 is a digital camera that is embedded within the smartphone 150. For instance, the image capture device 150 can be the front-facing digital camera that is integrated with the smartphone 800d and functionable as a component of the smartphone 800d at the time of its deployment. In this embodiment, the image capture device 150 can be easily used by the user 105, without requiring an external attachment, to simply take a close-up photo of the hair. In another example, the smartphone camera can be used for an initial hair image capture to receive preliminary instant recommendations.

As an example, user 105 can activate, or turn on the image capture device 150 for their smartphone 800d (e.g., via a mobile application installed thereon that implements the software application 140). Then, the user 105 can aim a lens of the image capture device 150 towards their head region at various angles, thereby obtaining one or more microscopic digital images of the user's 105 hair at a magnification level suitable for a hair strand to be visible by the naked eye. Furthermore, by capturing microscopic digital images of the user's 105 hair using the image capture device 150, the resulting digital images can be automatically submitted by the software application 140 on the smartphone 800d as hair samples to be analyzed by other components of the platform 100 (e.g., application modules 125) in order to detect the user's 105 hair texture, type, and condition and recommend hair care regimens including products, ingredients, techniques, and professional services. For example, a mobile application form of the software application 140 enables microscopic digital images of the user's 105 hair strand (obtained by image capture device 150) to be automatically uploaded to the remotely located server 110 via a communication network, such as Wide Area Network (e.g., the Internet), for further analysis. Microscopic digital images of the user's 105 hair can also be captured through use of other hardware device with a microscopic camera, which are similarly delivered to the other aspects of the platform 100 on the cloud for analysis. Hardware devices used for capturing microscopic digital images of the user's 105 hair may be deployed to be easily accessible to end users, for example being situated throughout the hair industry ecosystem, primarily in hair salons and beauty supply stores, and even personal homes to support easy at-home analysis.

It should be appreciated that capturing microscopic digital images via the image capture device 150 is not intended to be the only technique for providing hair samples of a user to the platform 100 for further ML-based hair analysis. A physical hair sample, for instance multiple actual hair strands combed from different regions of the user's head, can be submitted to the platform 100 as a hair sample. For example, as hair strands naturally shed from the head during routine hair care, such as combing the hair, brushing the hair, or washing the hair, these shedded hair strands (already removed from the user's scalp) can be physically submitted to the platform 100 as hair samples. Further, a SEM or imaging device can be used to scan and analyze these physical hair samples at the platform 100 (e.g., after the hair samples are received by the platform 100) or at the location of the user, where the images are then communicated to the platform 100 as hair samples. It should be appreciated that any of the aforementioned techniques for obtaining hair samples from a user 105 can be used singularly or in combination by the platform 100.

According to the embodiments, the software application 140 is configured to obtain determinations on one or more characteristics of the user's 105 hair based on the received hair sample (e.g., microscopic digital images of a hair stand). The one or more characteristics of hair can include, but are not limited to: 1) hair texture, 2) type, and 3) condition. Furthermore, based on the determinations of the user's hair characteristics, the software application 140 can obtain recommendations on hair care regimens that are particularly tailored for the user's 105 hair, including products, ingredients, techniques, and professional services. Therefore, the platform 100 provides an automated, accurate, efficient, and simple solution for users to identify their hair type and texture, discover the right hair products to use to give them the best results, receive a proper hair care regimen to follow, and receive accurate hair health and style guidance. According to the embodiments, other forms of recommendations that can be automatically provided by the platform 100 can include, but are not limited to: recommended salons and services; recommended styles, recommended hair stylists; recommended tutorials

Referring again to FIG. 1A, a block diagram of an operating environment consistent with the embodiments disclosed herein is illustrated. More specifically, FIG. 1A illustrates a block diagram of a hair assessment diagnosis and recommendation platform 100 consistent with the embodiments disclosed herein. FIG. 1A shows a user interface module 115 as a component of the software application 140. The user interface module 115 can be configured to provide the graphics, windows, I/O interfaces, and the like, which allows a user 105 to interact with the platform 100 via one or more electronic devices/computing systems 800a-800d. As alluded to above, the electronic devices 800a-800d that can employ the user interface module 115 includes but is not limited to a desktop 800a, laptop computer 800b, tablet computer 800c, and smartphone 800d. User 105 may utilize the user interface module 115 to control the microscopic image capture, such as activating the function of the image capture device 150, and to provide user data or input information in response to a questionnaire.

According to the embodiments, the user interface module 115 can includes websites for the user 105 to interact with and/or control the various functions of the platform 110. The websites may include a collection of user-facing landing pages and promotional tools that are used to inform customers, such as user 105, of hair care products, and the hair care recommendations automatically generated by the platform 100. The user interface module 115 can include a portal that is accessible to select staff members, in order to edit internal content and data used by the platform 100. All of the information that is input into the user interface module 115 (or otherwise communicated to the platform 100) is secure in accordance with the security features of the platform 100, which are described in greater detail below in reference to FIG. 1D.

Furthermore, the user interface module 115 can implement a mixture of the hybrid app framework with the React Native and native code to supplement platform dependencies. This allows applications of the software application 140 and the user interface module 115 to be compatible with different types of hardware platforms (e.g., Android™ and iOS™) with minimal configurations.

FIG. 1A also illustrates a central computing system 130 comprising a server 110 and a hair characteristics data module 120. FIG. 1A also illustrates a professional analysis module 165 having a data analyst specialist 145, analyzing hair sample (or specimen) data using an electronic device 850 and transmitting that analysis to the central computing system 130. Also seen in FIG. 1A, are external data databases 135 which provide more information for accurate analysis relating the hair sample data (also referred to herein as specimen data) based on data compiled, studies conducted, or research from libraries, medical professionals, hair specialists, or technicians.

In addition, FIG. 1A illustrates a series of application modules 125 which serve to utilize, service, and act on the provided information by the hair assessment diagnosis and recommendation platform 100. In the example of FIG. 1A, the application modules 125 includes: hair characteristics module 121 configured to determine the one or more characteristics of a user's hair based on the hair sample; machine learning module 122 configured to apply AI and ML technology for analysis of the hair; recommendation module 123 configured to provide hair care recommendations specific to the user and based on the determined hair characteristics (or attributes); hair products attributes module 124 configured to determine hair care products matching the hair characteristics that have been determined for the user; and predictive analysis module 126 configured to predict hair characteristics based on analyzing the user's hair over time.

In an embodiment, the hair characteristics module 121 can use the determined hair characteristics in order to derive an overall classification for the user's hair. The characteristics module 121 may apply a technique to classify the unique hair profile, by establishing a Unique Hair Identifier (ID) that corresponds to each user of the platform 100. The classification of hair profile, namely the Unique Hair ID, can serve as a universal standard and unique identifier to the user, so as to eliminate subjectivity and confusion that is associated with many existing hair charts and other hair classification approaches, which makes it even more difficult for a user to accurately identify their hair type, and store the recommendations for the Unique Hair ID over time and through multiple different retail and content channels. Further, the platform 100 can classify hair in an automated and efficient manner, which substantially improves accuracy over many existing (e.g., manual) hair classification approaches. For instance, the user or a hair stylist may visually inspect the hair using their naked eye, in order to classify the hair based on their personal and professional knowledge and their estimation of a suspected curl pattern. Although curl pattern may be easier for visual inspection by the human eye, it is not the only factor that determines the best products, and suggests a products reaction on the hair. Accordingly the characteristics module 121 considers several major attributes (not limited to curl pattern) in order to classify the hair, and ultimately generates an alphanumeric code that corresponds to the classification. Generally, the platform 100 uses the hair sample, such as the microscopic digital image of a hair strand, to detect and analyze the texture, type, and condition of the hair. These characteristics are further used to determine the Hair ID, which serves as a standardized form of classification for the hair. Thus, by employing the Hair ID as a type of classification, the platform 100 can successfully categorize a wide range of hair by texture, type, and condition.

In an embodiment, the characteristics module 121 analyzes six attributes, or characteristics of hair, including: type (curl pattern), texture (diameter, or circumference), health (condition), porosity, elasticity, and density (hairs on head). Thus, after analysis on each attribute is performed, a corresponding value is assigned to the particular characteristic. For example, hair analysis may determine that a hair sample has a diameter that is less 59 millimeters (mm), which causes the hair to be assigned a texture=A, indicating that the hair has a fine texture. The individual values for each of the six attributes can be aggregated to derive a six digit alphanumeric code for the hair classification, and an additional alphanumeric combination to capture values from the users questionnaire. The table 1 below includes examples of the attributes used in accordance with the disclosed hair classification techniques.

An as example of classifying a hair sample, the characteristics module 121 can assign a value to each of the attributes: type=3; texture=b; health=2; porosity=a; elasticity=2; density=c. Continuing with the example, the resulting Hair ID used to classify the hair in this example can be ID=3b2a2c, which is a code that is a composite of each value corresponding to each of the respective attributes. Subsequently, the Hair ID can be used to select one or more correlating recommendations using data science and cloud computing. For instance, after the user's 105 hair is classified by assigning it a Hair ID, the Hair ID can be input into the recommendation module 123 in order to determine one or more hair care recommendations that may be predefined as corresponding to that specific Hair ID, or hair classification. For example, based on characteristics of the user's hair indicated by the unique Hair ID and the user's needs, hair care products can be matched based on hair challenges, hair goals, and product performance attributes such as ingredients (where an ingredients analysis will be performed to understand what ingredients work well with what type/classification of hair).

Additionally, the embodiments can employ another set of alphanumeric characters, for example using data pulled from the questionnaire, in order to further personalize the unique Hair ID. Consequently, each resulting unique Hair ID can be completely personalized to the user such that no two users will have the same unique Hair ID (similar to social security numbers). An example of a unique Hair ID having the additional set of alphanumeric characters added to the aforementioned six-character ID is: 2a3d3a-CVM-87-ACDBCCD-1241-AB. In other words, the extended unique Hair ID provides a representation, and ultimately an assessment, for hair that is as unique as a fingerprint. The unique Hair ID is designed to identify the user throughout the hair industry ecosystem, and follow the user's hair journey and hair activity online and offline (e.g., stores, retailers, shops, salons, etc.) to make recommendations and further drive transactions. The unique Hair ID can also be used as an identifier that is tied to the electronic hair record as well. For example, the unique Hair ID can be used to pull up a particular user's hair record, from an Electronic Hair Records, system, in a manner similar to accessing and/or maintaining electronic health records in the realm of digital health.

As alluded to above, cloud computing may be employed such that microscopic digital images of the user's 105 hair can be communicated from the computer device, such as smartphone 800d, and stored at the server 110 for analysis by the application modules 125. In some embodiments, the machine learning module 122 can apply AI and ML algorithms to translate the digital images, captured as hair samples, into raw data which can then be used to automatically detect the hair characteristics and/or attributes, and classify the hair almost instantly. With obtaining a microscopic view of the hair strand (e.g., produced with a digital microscope) the shaft of the hair can be rendered by the image in high detail. This enables image analysis to be perform by the application modules 125. Image analysis performed on the microscopic digital image can include measuring the curl pattern, hair texture, diameter of the hair strand, cuticle layer, hair shaft, ends, detecting other particles, and recording other observations related to the hair shaft. For instance, the curl pattern of the hair strand captured by the microscopic digital image can be documented. Further, porosity and elasticity can be measured, and density detected as a result of image analysis. Subsequently, the AI and ML technology implemented by the hair characteristics module 125 and the hair care products attributes module 125 can function in concert to return hair care product matches that are most accurately paired with the profile attributes and hair data gathered from the hair analysis performed on the microscopic digital images of hair.

Additionally, the platform 100 can include the capability to apply forms of hair analysis, such as a hair mineral analysis, in order to provide health recommendations and health care regimens that further supplements the recommendations regarding hair care, or for their general health care independently. As background, hair analysis (e.g., analyzing and ascertaining a state of hair strands) can be used as a scientific approach to determine a long lasting record relating to the health of the user. For example, a person's hair strands can be analyzed to indicate certain elements (e.g., protein, heavy metals) that were in the blood of the person when the particular strand was forming. In some cases, hair analysis can be used to screen for tendencies for diabetes, heart disease, chronic fatigue, cancer and many other serious conditions. Hair analysis can also reveal diet, stress level, physical and mental health conditions, nutrition levels, toxicity, energy and vitality level, gland and organ insights, carbohydrate tolerance, trends or tendencies for a plethora of common health conditions, autonomic nervous system assessment, and psychological/emotional illness assessment.

Hair analysis may be a more accurate indicator than blood tests in some cases, as it shows trends over time, not just a snapshot. Thus, hair analysis is leveraged by the platform 100 in a manner that can track an overall health journey for a user 105, thereby providing a personalized approach that analyzes any changes of the hair over time for a particular user. In addition to hair care, the scientific and technical systems and methods implemented by the platform 100 can also use hair analysis to obtain general health data pertaining to the user. For instance, the platform 100 may determine that the user 105 has some nutrient deficiencies (e.g., iron, calcium) from their regular diet, by analyzing their provided samples of hair. Subsequently, the platform 100 can provide health care recommendations that are personalized for the user 105 regarding diet and nutrition that compensates for their deficiencies and promotes better general health. Consequently, the platform can 100 provide long-term support for a user 105, for instance analyzing the user's 105 hair type over several years that includes a personalized and holistic approach to improve the person's hair, health, and beauty.

FIG. 1B illustrates a block diagram of an operating environment consistent with the embodiments disclosed herein. More specifically, FIG. 1B illustrates a block diagram of a hair assessment diagnosis and recommendation platform 100B consistent with the embodiments disclosed herein. FIG. 1B, 115 shows a user interface module which allows a user 105 to interact with the platform via one or more electronic devices/computing systems 800 wherein the electronic devices include but is not limited to a desktop, laptop, tablet, or smartphone. User 105 may utilize the user interface module to control the image capture, manage the microscope attachment device, and provide user data or input information in response to a questionnaire. FIG. 1B, 150 illustrates a central computing system comprising a data analysis module 150B having a server 110 and a data communications module 150A. FIG. 1B also illustrates a professional analysis module having a data analyst specialist 145, analyzing specimen data using an electronic device 800 and transmitting that analysis to the central computing system 150. FIG. 1B, 135 illustrates external databases which provides more information for accurate analysis relating the specimen data based on data compiled, studies conducted, or research from libraries, medical professionals, hair specialists, or technicians. FIG. 1B, 155 illustrates a hair information database module which serve to utilize, service, and act on the provided information by the hair assessment diagnosis and recommendation platform. The hair information database module may further comprise additional application modules which include but are not limited to a hair characteristics module, a machine learning module, a recommendation module, a hair products attributes module, and a predictive analytics module.

FIG. 1C illustrates a block diagram of an operating environment consistent with the embodiments disclosed herein. More specifically, FIG. 1C illustrates a block diagram of a hair assessment diagnosis and recommendation platform 100C consistent with the embodiments disclosed herein. FIG. 1C, 115 shows a user interface module which allows a user 105 to interact with the platform via one or more electronic devices/computing systems 800 wherein the electronic devices include but is not limited to a desktop, laptop, tablet, or smartphone. User 105 may utilize the user interface module to control the image capture, manage the microscope attachment device, and provide user data or input information in response to a questionnaire. FIG. 1C illustrates a central computing system comprising a server 110. FIG. 1C, 160 illustrates a services module including but not limited to a predictive analytics module, an evaluation module, a simulation module, a professional analysis module and transmitting that service module information to the central computing system 110. The simulation module may provide the user with simulated potential hair outcomes, hair styles, and/or hair assessments. FIG. 1C, 170 illustrates datastores including but not limited to hair data records, hair analysis records, recommended assessments, and recommended styles. These datastores may also include additional databases or be connected with external databases which may provide more information for accurate analysis relating the specimen data based on data compiled, studies conducted, or research from libraries, medical professionals, hair specialists, or technicians. FIG. 1C, 165 illustrates a series of application modules which serve to utilize, service, and act on the provided information by the hair assessment diagnosis and recommendation platform. Application modules include but are not limited to a hair characteristics module, a machine learning module, and a recommendation module.

As briefly described above, the present disclosure relates, in various aspects, to a hair assessment diagnosis and recommendation platform. In one aspect, the present disclosure provides Scientific and Technical Systems and Methods for Providing Hair Health Assessment and Styling Recommendations. In further aspects, the present disclosure relates to a data capturing method. In still further aspects, the present disclosure relates to an image capturing method. In even further aspects, the present disclosure relates to a scientific analysis method and system. In yet even further aspects, the present disclosure relates to a relational database method and system. In still other aspects, the present disclosure relates to a recommendation method and system.

In further aspects, the disclosed devices provide a hair health assessment diagnosis and recommendation platform. In still further aspects, the device can be useful for providing Scientific and Technical Systems and Methods for Providing Hair Health Assessment and Styling Recommendations. In even further aspects, the disclosed devices may provide for scientific data collection and analysis.

Referring now to FIG. 1D, another diagram of the Hair Health Assessment Diagnosis and Recommendation Platform 100 is shown. Particularly in FIG. 1D, the multiple layers 905-930 of a technology stack 900 implementing the various functions and features of the platform 100 are illustrated. Interfaces and multiple layers of the technology stack 900 are designed to make the user experience to be as seamless as possible. The platform 100 is built to provide privacy, scalability, and security as a primary focus. As seen in FIG. 1D, the layers of the technology stack 900 can include, but are not limited to: User Account Management Layer 905; Customer Relationship Management Layer 910; AI-based recommendation Engine Layer 915; Internal API management Layer 920; API Integration Layer 925; and Security Layer 930.

The User Account Management Layer 905 can be implemented via a user session management module, where the module is used for handling new user processes like user sign-up, sign-in, register, and handling of any onboarding sessions that require collecting required personal identifiable information (PII) securely through Secure Sockets Layer (SSL) API calls towards cloud databases. The User Account Management 905 aspects can also implement a user account management module that enables further processing of new users, usually for the purpose of sales, promotion and customer follow-up, by automatically handing off new data to an API at the Customer Relationship Management layer 910.

The Customer Relationship Management (CRM) Layer 910 can implement APIs that enable a new user's information to get handed off (to customer relationship management API) as soon as onboarding and registration data points are met for further processing. During onboarding, the user's information is collected, such as the user's first name, last name, primary address, email, phone number and a brief statement from our users that encourages them to explain what their “Perfect hair day” is in their own words through one or two paragraphs. Then, the data that is collected gets automatically entered into an independent third party CRM tool, so that the customer service staff can easily view and welcome each customer through automated email and one-on-one sessions. The user also has the capability of requesting contact through apps as needed for consultation with their chosen method, either by phone, video, or email with a customer service representative who then further qualifies users that need to have their questions answered with in-house hair experts. It should be understood that the flow of user onboarding implemented by the Customer Relationship Management Layer 910 is subject to change through many factors (e.g., new company partnerships). As a result, the module for the Customer Relationship Management Layer 910 can be configured to collect appropriate information by recognizing leads from campaign emails for certain products and partnerships.

The AI-based Recommendation Engine Layer 915 can implement the AI and ML based core of the application features, which allows the platform 100 to continuously improve its functionality in a manner that allows users to be able to upload a self-portrait or a “Selfie” to an image classifier, where AI/ML coupled with other filtration steps, provides a response that identifies the user's hair type into multiple hair categories. For example, the AI-based Recommendation Engine Layer 915 is configured to detect whether the user has curly, straight, wavy, coily, or kinky hair and the percentage combination of each using automation. Then, the AI-based Recommendation Engine Layer 915 passes on the attributes to the recommendation engine to provide products that will be appropriate for that hair type. By employing a growing data set of hair products, the data points that are collected from recognizing the image file of the self-portrait (e.g., smartphone image of the hair) and the recommendation engine that filters out appropriate products can be updated or altered individually to increase the accuracy of the AI-based Recommendation Engine Layer 915.

The Internal API Management Layer 920 can include internal APIs hosted on several Elastic (EC2) instances on Web Services, designed for scale and high bandwidth requests. In some embodiments, the internal API calls are documented for developers in a very simple and easy to understand format. Custom API documentation may be available for outside vendors or partners upon request, due to its potentially dynamic nature and the security of the application for each corporate clients' needs. Third-party APIs can also be implemented at the Internal API Management Layer 920 for some non-proprietary features, for instance marketing and analytic services.

The API Integration Layer 925 can be configured to support on-boarding and promotions. For example, by building the API Integration Layer 925 solely for on-boarding and promoting sponsor's brands fairly, the timeframe it takes to provide a tailored user a sponsored experience can be minimized. Clients can range from “product-centric” to “information and content-centric” so it may be important to have onboarding procedures that are relevant to each of our corporate clients that are supported by the API Integration Layer 925.

FIG. 1D also shows that the Security Layer 930 can be included in the technology stack 900. The platform 100 can secure its systems through custom AWS security group rules and through VPN access for developers to deploy new live builds. Amazon™ Web Services (AWS) security groups (SGs) are associated with EC2 instances and can provide security at the protocol and port access level. Each rule implemented at the Security Layer 930 can contain a set of rules that filter traffic coming into and out of our host API layers. The Security Layer 930 may be configured to may be proactively check the platform 100 for vulnerabilities through scheduled bi-quarterly, third-party penetration testing services. Occasionally, when there is a major feature update to the platform 100 that requires us to review new end-points, the Security Layer 930 can also perform the proactive checks. The Security layer also protects Personally Identifiable Information (PII) and is HIPAA compliant.

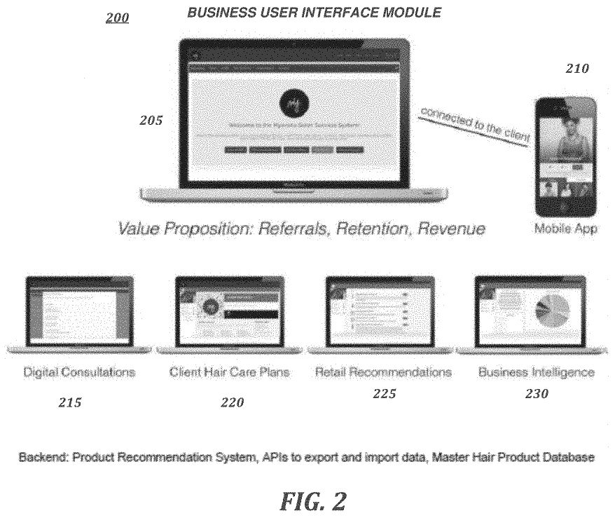

FIG. 2 illustrates a business user interface module consistent with the embodiments disclosed herein. More specifically, FIG. 2, 200 illustrates a business user interface module which allows business owners or business customers using the platform to manage their data and user experience for their clients. FIG. 2, 205 illustrates an embodiment of a user interface module welcome screen. FIG. 2, 210 illustrates an embodiment of a client user interface module on a mobile device. FIG. 2, 215 illustrates an embodiment of a user interface module digital consultations screen. FIG. 2, 220 illustrates an embodiment of a user interface module for personalized hair care plans screen. FIG. 2, 225 illustrates an embodiment of a user interface module retail recommendations screen. FIG. 2, 230 illustrates an embodiment of a user interface module business intelligence screen. The business user interface module is not limited to the screens found in FIG. 2. this platform 100.

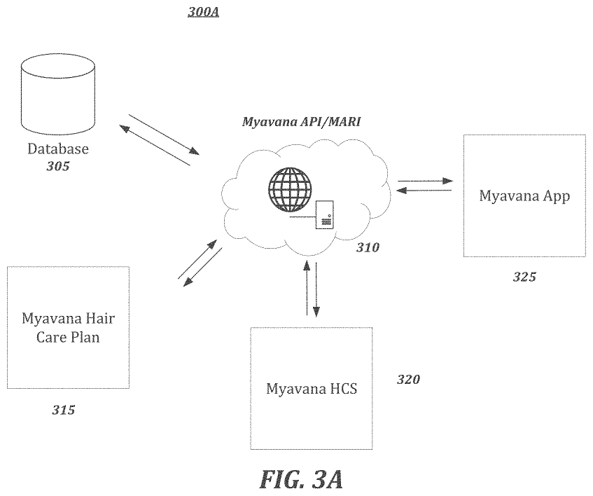

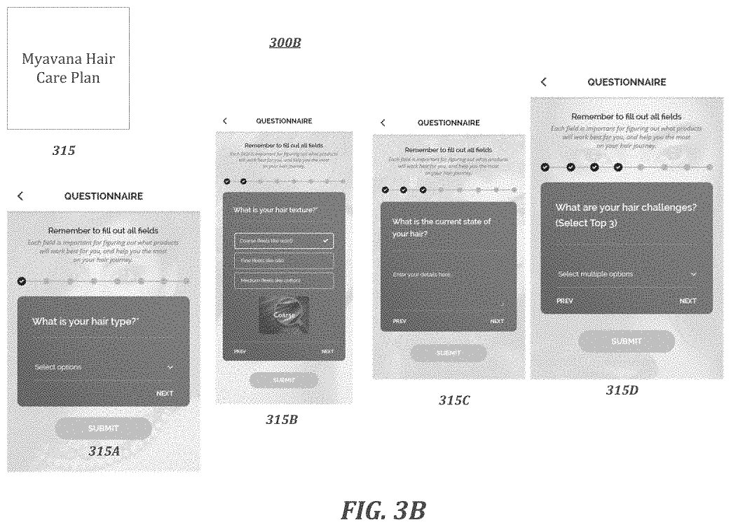

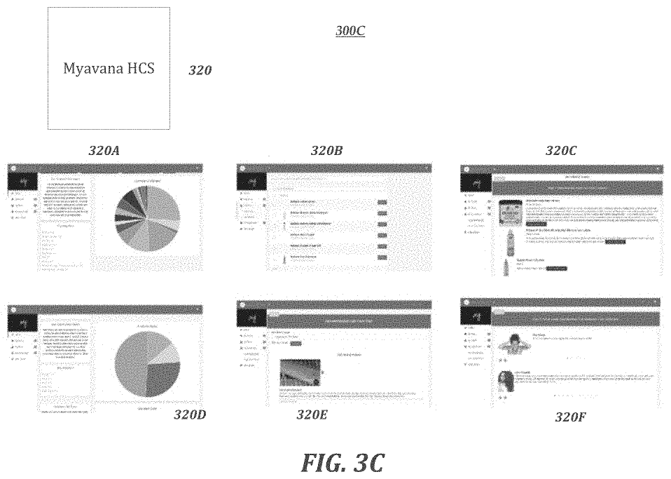

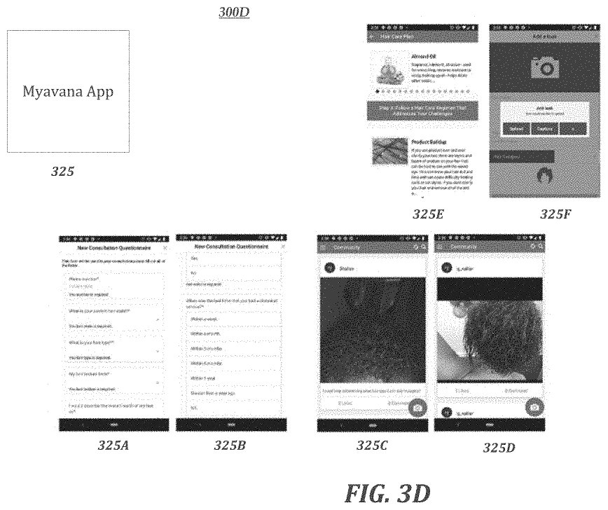

FIG. 3A shows a depiction of an application programing interface (API) and operating systems in accordance with an embodiment of the present disclosure. More specifically, FIG. 3A, 300A illustrates an infrastructure or a system supporting the hair assessment diagnosis and recommendation platform. FIG. 3A, 305 illustrates a database which may be used as an element of the system supporting the hair health assessment diagnosis and recommendation platform 100. The database 305 may include but not be limited to an Amazon Web Services (AWS), Amazon Relational Database Service (RDS), Microsoft Azure, Google Cloud, Apple iCloud, and the like. FIG. 3A, 310 illustrates a platform application programing interface (API) having a server, which may function as a cloud or central computing database. The API 310 may maintain, receive, and process all information driven and processed by the infrastructure held together and supported by the API 310. The API 310 may save and query information to and from database 305. FIG. 3A, 315 illustrates a personalized hair care plan module of the platform. The personalized hair care plan module 315 may be configured to process user input. The user input may be processed by the personalized hair care plan module 315 in order to analyze, determine, process, and deliver recommendations to one or more users. Recommendations provided by the hair care module 315 in various presentation formats. FIG. 3A, 320 illustrates a hair care system module. Hair care system module 320 may be configured to be used as a business portal. The hair care system module 320 infrastructure may be further configured to enable business users to manage team, manage inventory, and manage customer records. Hair care system module 320 may be further adapted to display analytical data from the hair assessment diagnosis and recommendation platform 100. FIG. 3A, 325 illustrates a platform software application. Platform software application 325 may be enabled as but not limited to: a social media application, a user interface module, a user experience portal, and a user feedback tool. Platform software application 325 may further be adapted to collect data and user information that may be used by the hair assessment diagnosis and recommendation platform 100 to analyze, determine, process, and deliver recommendations to one or more users. Platform software application 325 may further be adapted to deliver content and assessment regiments to users on their mobile devices 800.

FIG. 3B shows a depiction of a personalized hair care plan system in accordance with an embodiment of the present disclosure. FIG. 3B, 300B illustrates a personalized hair care plan system comprising a personalized hair care plan module 315 of the platform 100. The personalized hair care plan module 315 may be configured to process user input. FIG. 3B, 315A-D illustrate an embodiment of a questionnaire designed to get feedback and information from a customer. FIG. 3B, 315A illustrates a questionnaire with the sample question “What is your hair type?” FIG. 3B, 315B illustrates a questionnaire with the sample question “What is your hair texture?” FIG. 3B, 315C illustrates a questionnaire with the sample question “What is the current state of your hair?” FIG. 3B, 315D illustrates a questionnaire with the sample question “What are your hair challenges?” The personalized hair care plan module 315 may be configured to provide a variety of information, questions, or other types of prompts to initiate user input and user feedback. The user input may be processed by the personalized hair care plan module 315 in order to analyze, determine, process, and deliver recommendations to one or more users. Recommendations provided by the hair care module 315 in various presentation formats.

FIG. 3C shows a depiction of a hair care system in accordance with an embodiment of the present disclosure. FIG. 3C, 300C illustrates a hair care system comprising a hair care module system 320. Hair care system module 320 may be configured to be used as a business portal. The hair care system module 320 infrastructure may be further configured to enable business users to manage team, manage inventory, and manage customer records. Hair care system module 320 may be further adapted to display analytical data from the hair health assessment diagnosis and recommendation platform 100. FIG. 3C, 320A-F illustrate an embodiment of various business user interface module screens configured to enable business users to manage team, manage inventory, and manage customer records. Hair care system module 320 may be adapted to show hair products, ingredients, characteristics, side effects, uses, hair image data, customer database, customer comments, and other such information.

FIG. 3D shows a depiction of a hair care software application in accordance with an embodiment of the present disclosure. FIG. 3D, 300D illustrates a platform software application system. Platform software application 325 may be enabled as but not limited to: a social media application, a user interface module, a user experience portal, and a user feedback tool. Platform software application 325 may further be adapted to collect data and user information that may be used by the hair assessment diagnosis and recommendation platform 100 to analyze, determine, process, and deliver recommendations to one or more users. FIG. 3D, 325A-F illustrate an embodiment of various user interface module screens configured to enable users to perform actions including but not limited to: engage a new consultation questionnaire, share photos and/or messages with members of the software application 325 community, capture images, view assessment regimens, view hair care products, view hair care ingredients. Platform software application 325 may further be adapted to deliver content and regimens to users on their mobile devices 800.

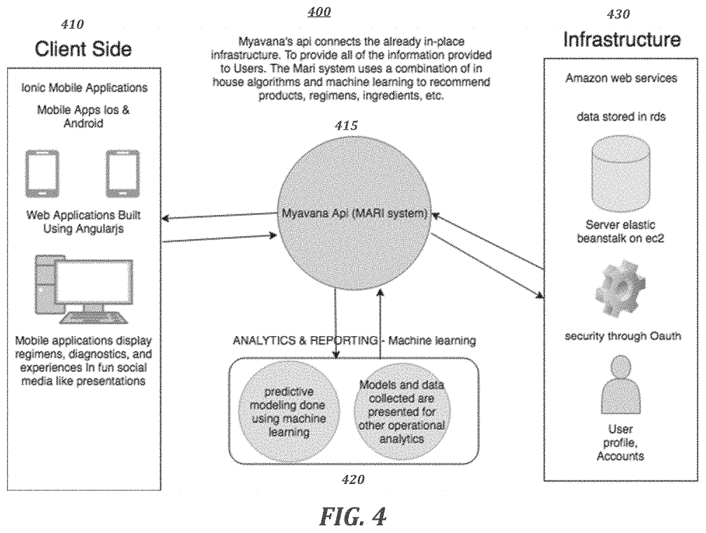

FIG. 4 shows a depiction of an operating system infrastructure in accordance with an embodiment of the present disclosure. More specifically, FIG. 4, 400 illustrates a detailed operating system infrastructure comprising an API (MARI system) 415, a client side 410, an analytics and reporting machine learning module 420, and an infrastructure 430. FIG. 4, 400 shows the operating system infrastructure having an API (MARI System) 415 which connects existing in-place infrastructure. API (MARI System) 415 may be configured to provide all information provided to users. The MARI system component of the API 415 may be configured to use a combination of in-house algorithms and machine learning to recommend products, regimens, assessments, ingredients, styles, and the like. The client side 410 comprising mobile applications, including but not limited to, iOS and Android, web applications including but not limited to web apps constructed using Angularjs. The client side 410 may further be configured such that mobile applications may display regimens, assessments, diagnostics, styles, and experiences in a presentation format similar to an enjoyable social media format. The analytics and reporting machine learning module 420 may be configured to provide predictive modeling done using machine learning. The analytics and reporting machine learning module 420 may be configured to model information, collect data, and present models and data to be processed by other operational analytics, and the like. The infrastructure 430 comprises databases which may include but not be limited to data stored using Amazon Web Services (AWS), Amazon Relational Database Service (RDS), Microsoft Azure, Google Cloud, Apple iCloud, and the like. The infrastructure 430 further comprises a server which may be configured to run using Amazon Web Services elastic beanstalk, ec2 and the like. The infrastructure 430 further comprises security elements including but not limited to security through token-based authentication, open authorization (OAuth). The infrastructure 430 further comprises user profile, and user accounts.

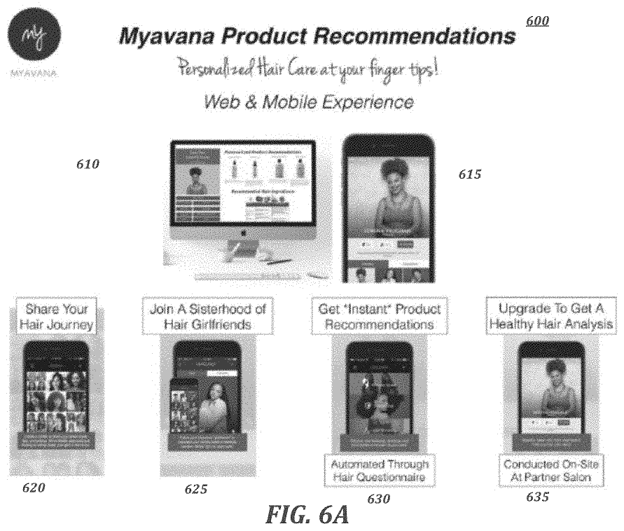

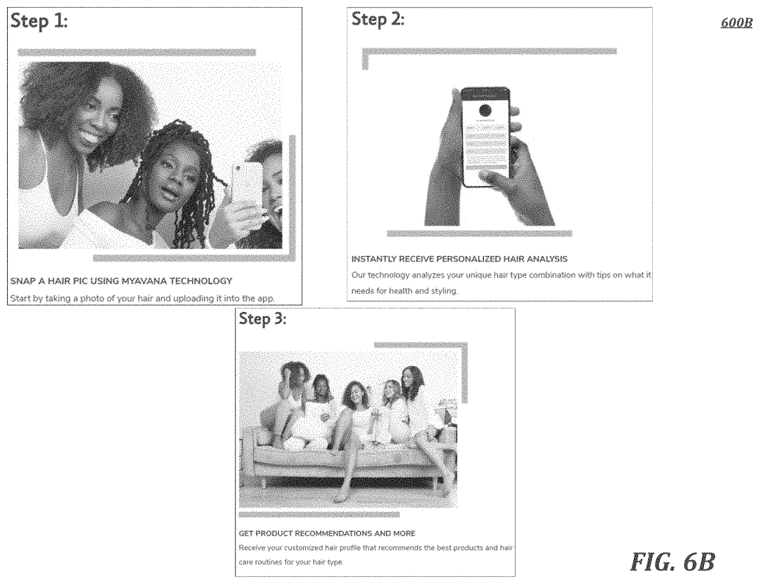

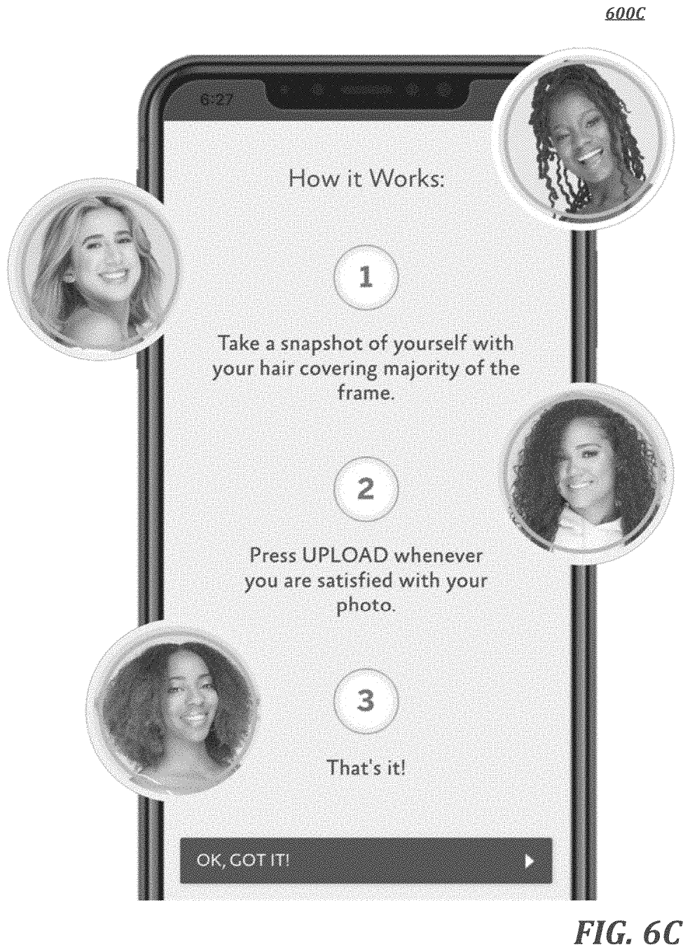

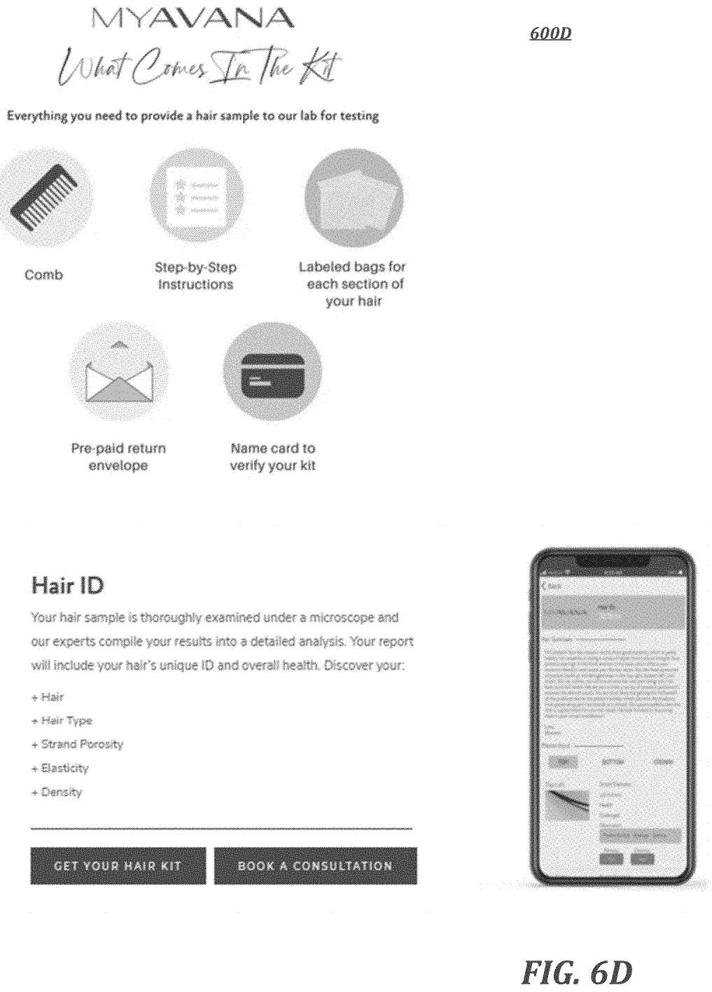

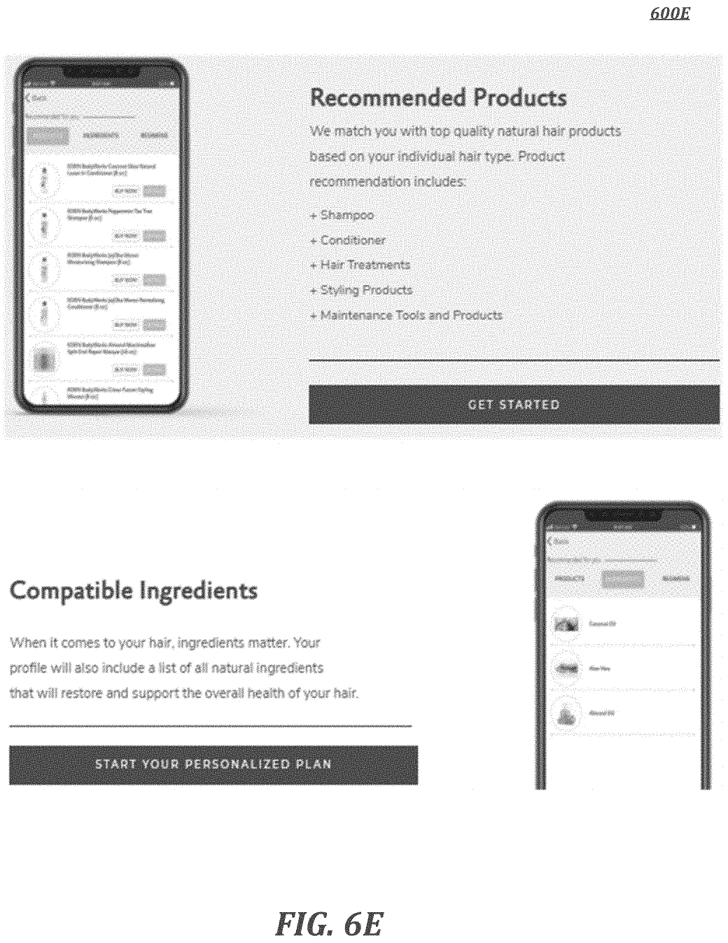

FIG. 6A shows a depiction of hair care software application embodiment for providing Scientific and Technical Systems and Methods for Providing Hair Health Assessment and Styling Recommendations in accordance with an embodiment of the present disclosure. More specifically, FIG. 6A shows a depiction of a recommendation system in accordance with an embodiment of the present disclosure. FIG. 6A, 600 illustrates an embodiment platform software application system. FIG. 6A, 610 illustrates a desktop platform and a mobile platform 615. Platform software application 600 may be implemented on a mobile platform 615. FIG. 6A, 620 illustrates a social media application function which may be configured to allow a user to share their hair journey; FIG. 6A, 625 illustrates a social media application function which may be configured to allow a user to join a community or sisterhood of hair girlfriends; FIG. 6A, 630 illustrates a platform software application which may be configured to provide real-time product recommendations or answer an automated hair questionnaire; FIG. 6A, 635 illustrates a platform software application which may be configured to allow a user to purchase a healthy hair analysis kit, capture a hair image, or schedule a hair & scalp analysis at an on-site salon. FIG. 6A, 600 illustrates an embodiment of various user interface module screens configured to enable users to perform actions including but not limited to: engage a new consultation questionnaire, share photos and/or messages with members of the software application 620, 625, 630, 635 community, capture images, view assessment regimens, view hair care products, view hair care ingredients, view hairstyles, view recommended professional hair stylist. Platform software application 600 may further be adapted to deliver content and assessment regimens to users on their mobile devices 800.

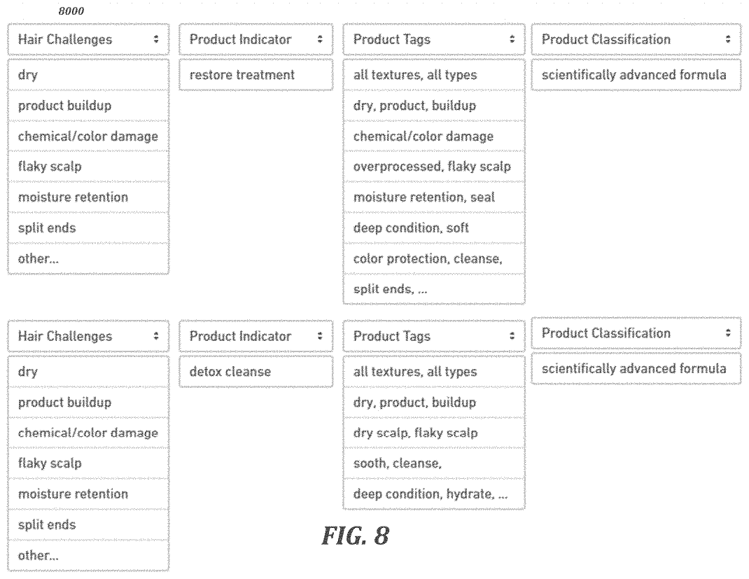

As will be detailed with reference to FIG. 8 below, the computing device through which the platform may be accessed may comprise, but not be limited to, for example, a desktop computer, laptop, a tablet, or mobile telecommunications device. Though the present disclosure is written with reference to a mobile telecommunications device, it should be understood that any computing device may be employed to provide the various embodiments disclosed herein.

FIG. 6B, 600B shows a depiction of a method for providing Scientific and Technical Systems and Methods for Providing Hair Health Assessment and Styling Recommendations in accordance with an embodiment of the present disclosure. In one or more embodiments as shown in FIG. 6B, a user may snap a hair picture using the hair care software application embodiment. Following taking the hair picture, the user may upload the hair picture to the hair care software application. The hair care software application embodiment may analyze the hair picture, conduct a hair health assessment, and deliver data including but not limited to a personalized hair analysis to the user. The personalized hair analysis may include a user's unique hair type combination with tips on what the hair needs for health and styling. After delivering the personalized hair analysis, the hair care software application may send hair product recommendations, hair care regimens, and routines that match the personalized hair analysis.

FIG. 6C, 600C shows a depiction of a method for providing Scientific and Technical Systems and Methods for Providing Hair Health Assessment and Styling Recommendations in accordance with an embodiment of the present disclosure. As depicted at least in FIG. 6B, FIG. 6C shows more detailed instructions for a user to snap a hair picture positioning the user's hair such that it covers the majority of the digital picture frame using the hair care software application embodiment. Following taking the hair picture, the user may uploading the hair picture to the hair care software application using an upload button or other input, gesture, movement, or the likeness thereof.

FIG. 6D, 600D and FIG. 6E, 600E shows a depiction of a hair care software application embodiment for providing Scientific and Technical Systems and Methods for Providing Hair Health Assessment and Styling Recommendations in accordance with an embodiment of the present disclosure. FIG. 6D, 600D shows an embodiment wherein a user may physically submit a hair sample to Myavana for lab testing. In one or more instances, more detailed lab testing of hair may be preferred or necessary. In this instance, a hair sampling kit comprising a comb, step-by-step instructions, labeled bags for each section of hair, name card for verification, and an envelope are provided to the user for providing hair samples and sending them to the Myavana laboratory. Upon thorough examination of the hair sample under a microscope coupled with expert analysis, the user will be provided with a detailed report. The report comprises a unique Hair ID and analysis results including but not limited to: hair health, hair type, strand porosity, elasticity, density, and other hair characteristics. The report may be provided to the user via the hair care software application embodiment for providing Scientific and Technical Systems and Methods for Providing Hair Health Assessment and Styling Recommendations. FIG. 6E, 600E shows an embodiment of the hair care software application embodiment for providing Scientific and Technical Systems and Methods for Providing Hair Health Assessment and Styling Recommendations. Initially providing recommendations matching top quality natural hair products based on the individual hair type including but not limited to recommended: shampoos, conditioners, hair treatments, styling products, maintenance tools, maintenance products, and the like. FIG. 6E, 600E also depicts providing a user with a compatible ingredients list which provides a list of all natural ingredients that will restore and support the overall health of the user's hair.

FIGS. 8-27 show a depiction of a graphical user interfaces (GUIs) that may be presented by the hair care software application embodiment for providing Scientific and Technical Systems and Methods for Providing Hair Health Assessment and Styling Recommendations in accordance with an embodiment of the present disclosure. FIG. 8, 8000 shows a graphical user interface (GUIs) that may be presented by the hair care software application embodiment indicating classification of various hair challenges, the associated product indicator, product tags, and product classification. The hair challenges including but not limited to: dryness, product buildup, chemical damage, color damage, flaky scalp, split ends, moisture retention, and other challenges. The product indicator including but not limited to: restorative treatment, detoxification cleanse treatment, and other treatments. The product tags including but not limited to: all textures, all types, dry, product buildup, chemical damage, color damage, overprocessed, flaky scalp, moisture retention, seal, deep condition, soft, color protection, cleanse, split ends, sooth, cleanse, hydrate, and other product tags. The product classification including but not limited to: scientifically advanced formula, and other classifications.

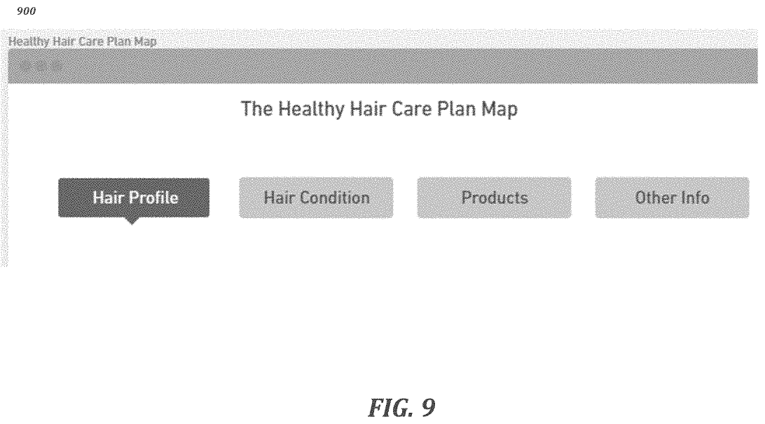

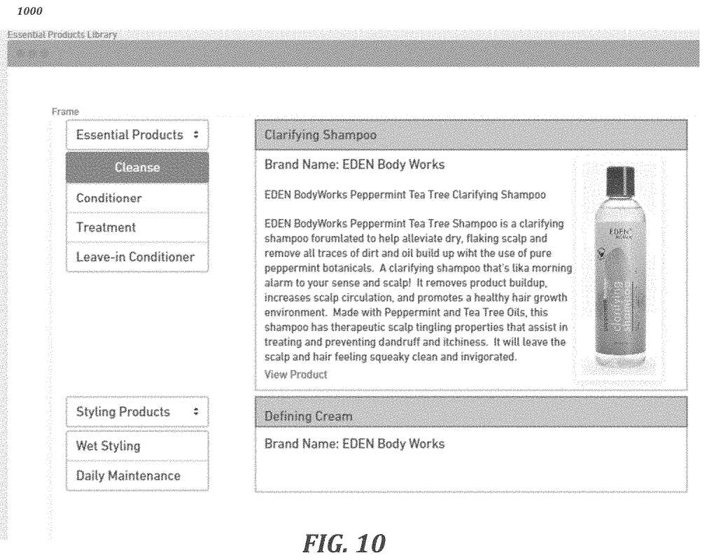

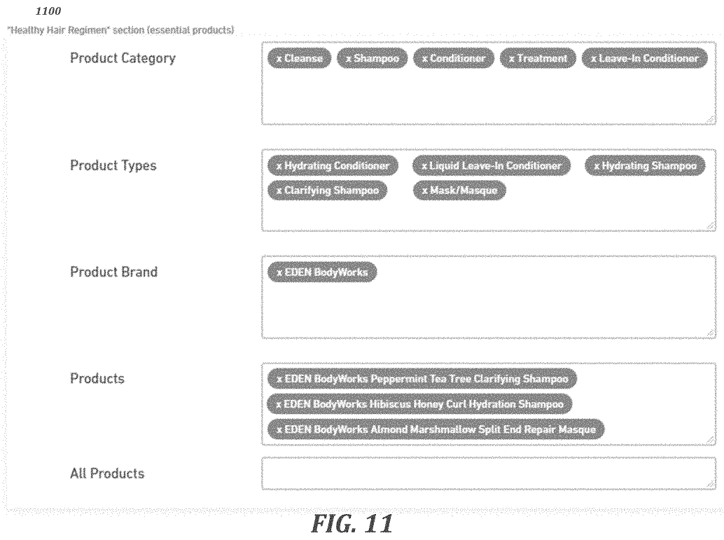

FIG. 9, 900 shows a graphical user interface (GUIs) that may be presented by the hair care software application embodiment indicating the healthy hair care plan including but not limited to: hair profile, hair condition, products, and other information. FIG. 10, 1000 shows a graphical user interface (GUIs) that may be presented by the hair care software application embodiment indicating the essential products library including but not limited to: various essential products. FIG. 11, 1100 shows a graphical user interface (GUIs) that may be presented by the hair care software application embodiment indicating the healthy hair regimen including but not limited to: product category, product types, product brand, products, all products, and other information.

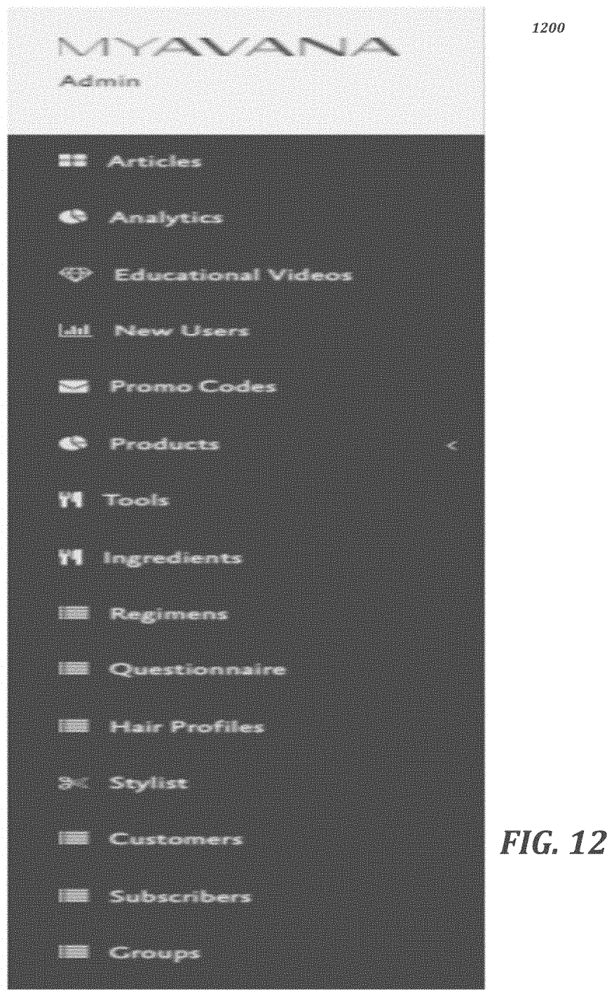

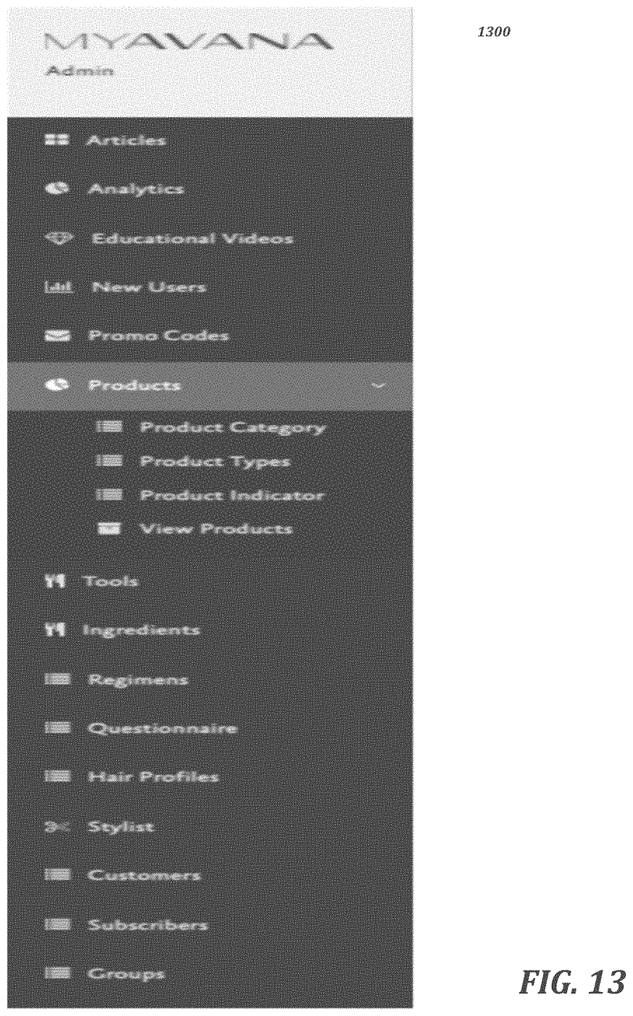

FIG. 12, 1200 shows a graphical user interface (GUIs) that may be presented by the hair care software application embodiment indicating an administrative panel including but not limited to: articles, analytics, educational videos, new users, promo codes, products, tools, ingredients, regimens, questionnaire, hair profiles, stylist, customers, subscribers, groups, and other information. FIG. 13, 1300 shows a graphical user interface (GUIs) that may be presented by the hair care software application embodiment indicating an administrative panel products including but not limited to: product category, product types, product indicator, view products, and other information.

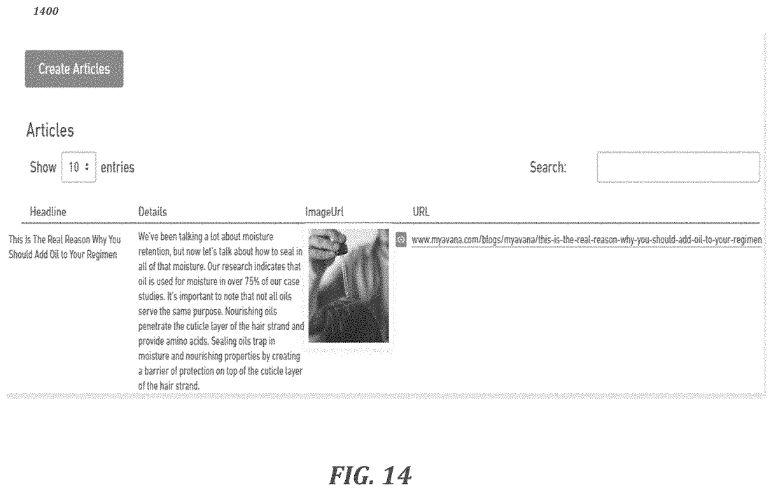

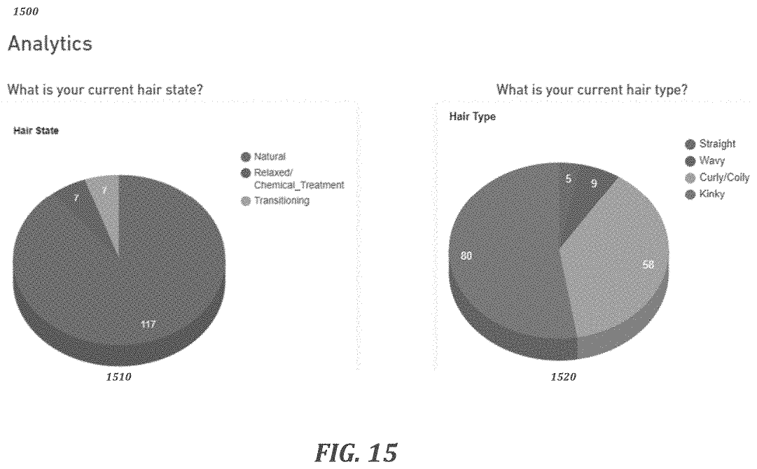

FIG. 14, 1400 shows a graphical user interface (GUIs) that may be presented by the hair care software application embodiment depicting the ability to create articles. FIG. 15, 1500 shows a graphical user interface (GUIs) that may be presented by the hair care software application embodiment depicting sample analytics. FIG. 15, 1510 shows analytical data for users current hair state. The data is depicted as a pie chart showing choices of natural, relaxed/chemical treatment, transitioning or other hair states. FIG. 15, 1520 shows analytical data for users current hair type. The data is depicted as a pie chart showing choices of straight, wavy, curly, coily, kinky, or other hair types.

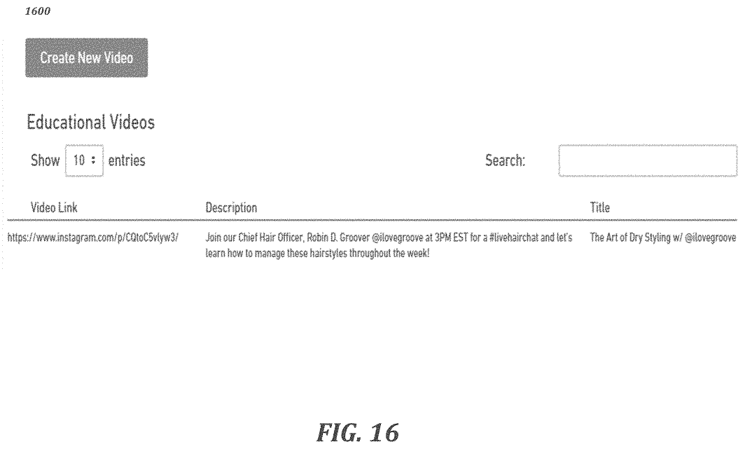

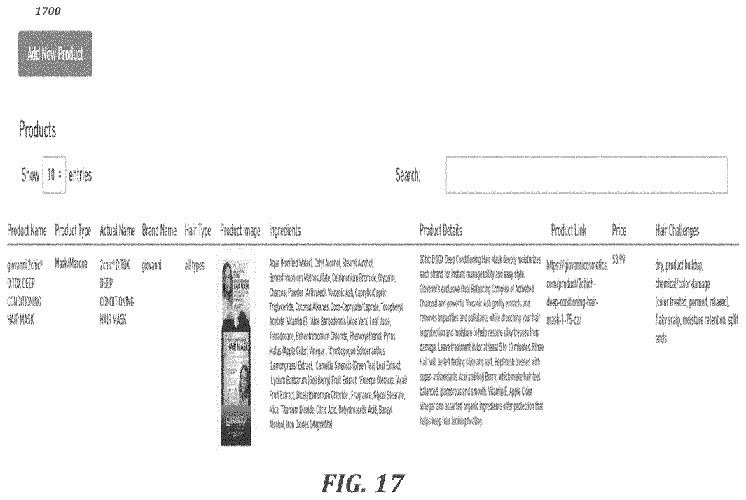

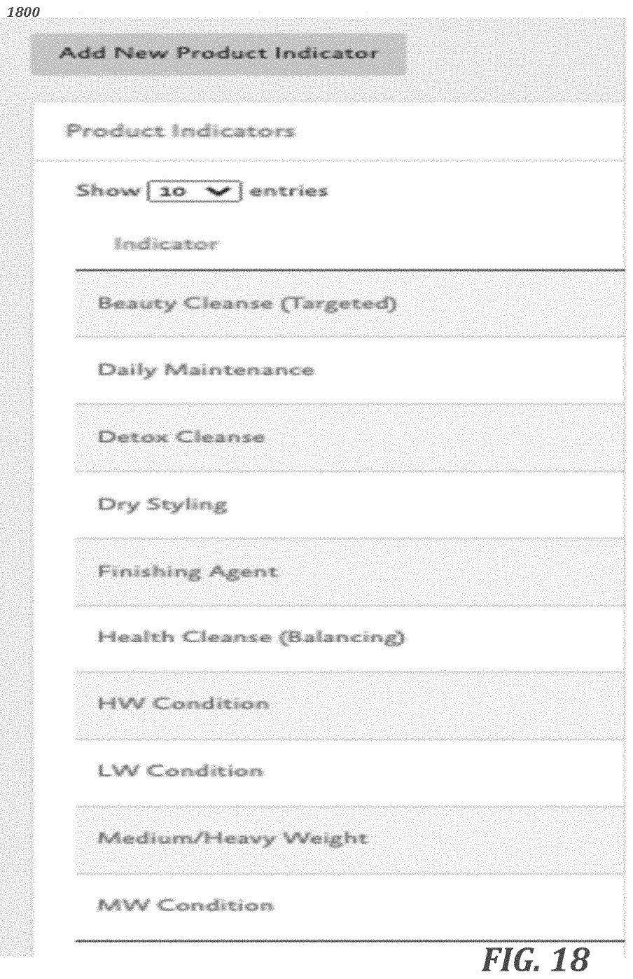

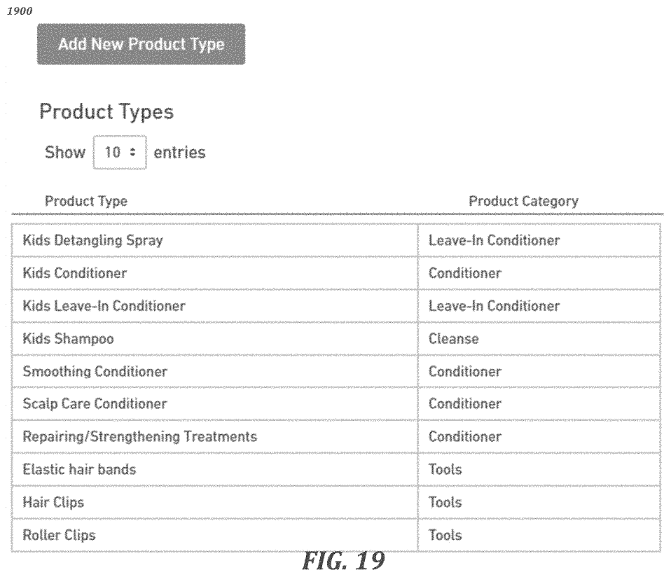

FIG. 16, 1600 shows a graphical user interface (GUIs) that may be presented by the hair care software application embodiment depicting the ability to create videos. FIG. 17, 1700 shows a graphical user interfaces (GUIs) that may be presented by the hair care software application embodiment depicting the ability to add new products. FIG. 18, 1800 shows a graphical user interface (GUIs) that may be presented by the hair care software application embodiment depicting the ability to add new product indicators, the product indicators including but not limited to: beauty cleanse (targeted), daily maintenance, detox cleanse, dry styling, finishing agent, health cleanse (balancing), heavy weight (HW) condition, light weight (LW) condition, medium weight, heavy weight, medium weight condition, and other product indicators. FIG. 19, 1900 shows a graphical user interface (GUIs) that may be presented by the hair care software application embodiment depicting the ability to add new product types with associated product categories. The product types may include the following but are not limited to: kids detangling spray, kids conditioner, kids leave-in conditioner, kids shampoo, smoothing conditioner, scalp care conditioner, repairing treatments, strengthening treatments, elastic hair bands, hair clips, roller clips, and other product types. The product categories may include the following but are not limited to: leave-in conditioner, conditioner, cleanse, tools, and other product categories.

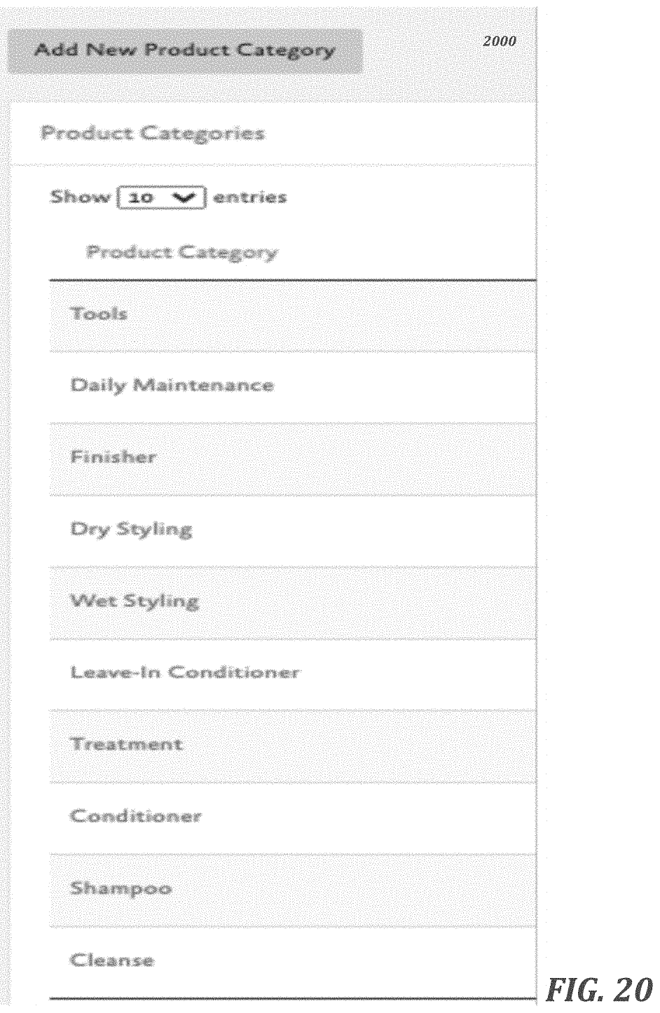

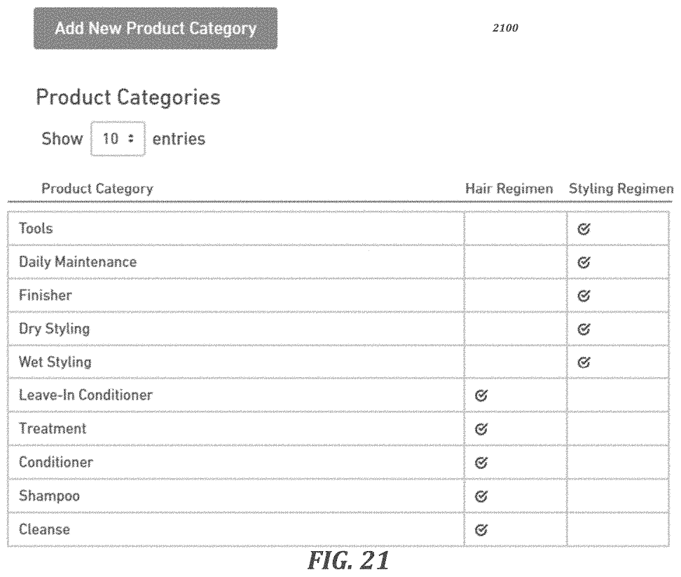

FIG. 20, 2000 shows a graphical user interface (GUIs) that may be presented by the hair care software application embodiment depicting the ability to add new product categories. The product categories may include the following but are not limited to: tools, daily maintenance, finisher, dry styling, wet styling, leave-in conditioner, treatment, conditioner, shampoo, cleanse, and other product categories. FIG. 21, 2100 shows a graphical user interface (GUIs) that may be presented by the hair care software application embodiment depicting the ability to add new product categories with associated regimen classifications. The product categories may include the following but are not limited to: tools, daily maintenance, finisher, dry styling, wet styling, leave-in conditioner, treatment, conditioner, shampoo, cleanse, and other product categories. The regimen classifications may include the following but are not limited to: hair regimens, styling regimen, and other regimens.

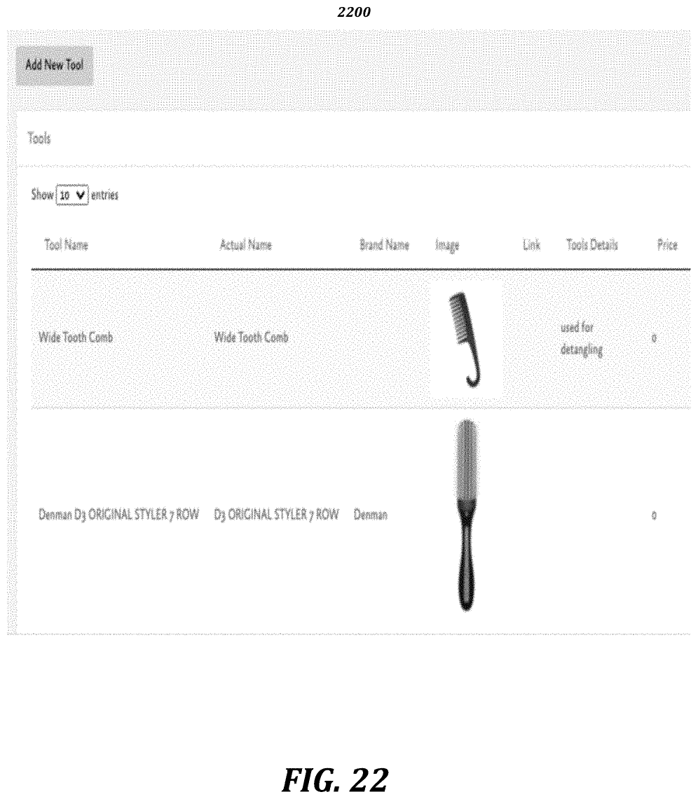

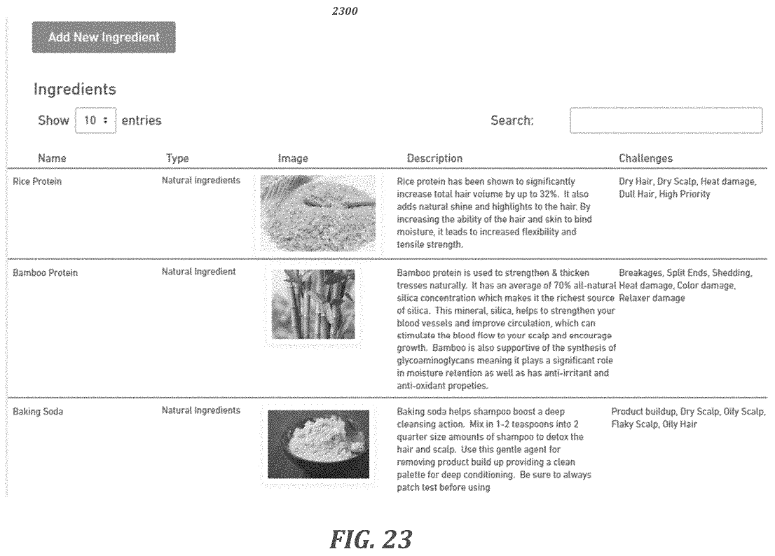

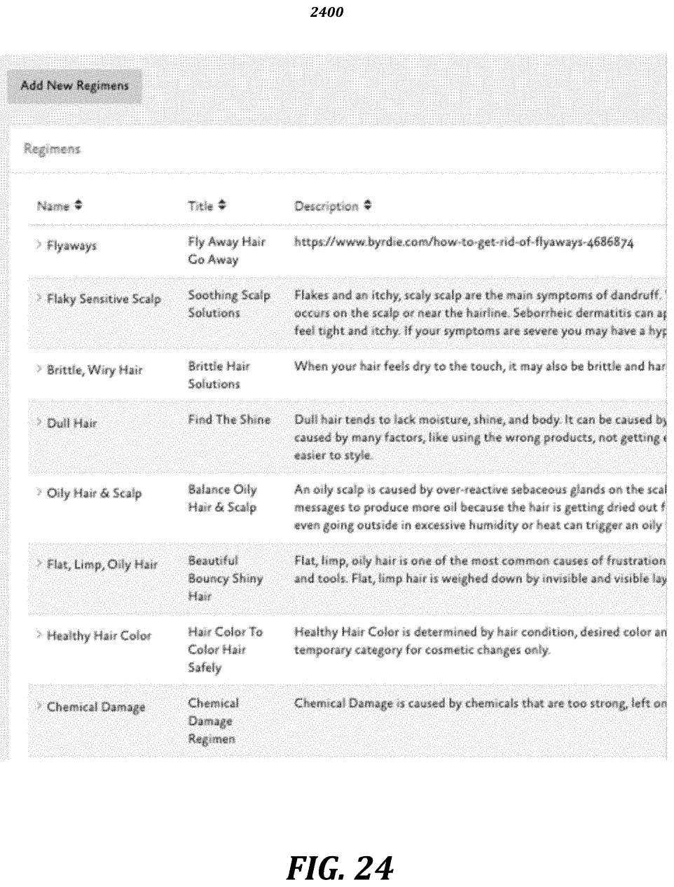

FIG. 22, 2200 shows a graphical user interface (GUIs) that may be presented by the hair care software application embodiment depicting the ability to add new tools. The tools may include the following additional information but not limited to: tool name, actual name, brand name, image, link, tool details (description of uses and the like), pricing, and other relevant tool information. FIG. 23, 2300 shows a graphical user interface (GUIs) that may be presented by the hair care software application embodiment depicting the ability to add new ingredients. The ingredients may include the following additional information but not limited to: name, type, image, description, challenges, and other relevant ingredient information. FIG. 24, 2400 shows a graphical user interface (GUIs) that may be presented by the hair care software application embodiment depicting the ability to add new regimens. The regimens may include the following information but not limited to: regimen name, regimen title, regimen description, and other relevant regimen information. The regimens may include the following additional information but not limited to: flyaways, flaky sensitive scalp, brittle, wiry hair, dull hair, oily hair & scalp, flat, limp, oily hair, healthy hair color, chemical damage.

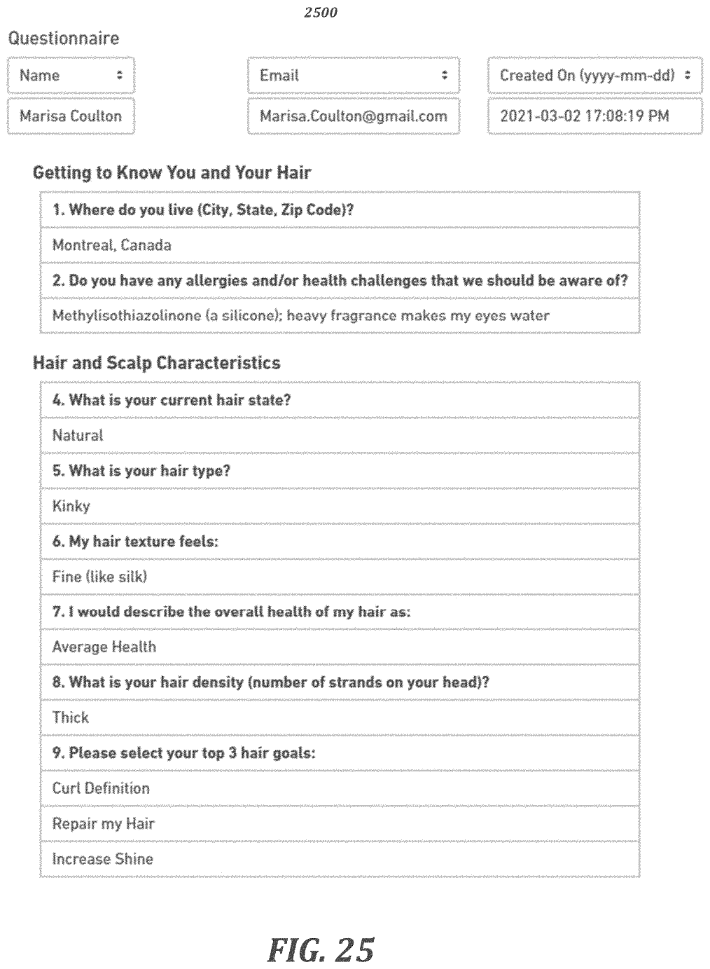

FIG. 25, 2500 illustrates an embodiment of a questionnaire designed to get feedback and information from a customer. FIG. 25, 2500 illustrates a questionnaire titled “Getting to Know You and Your Hair” and “Hair and Scalp Characteristics” with the sample questions: “Where do you live (City, State, Zip Code)?”; “Do you have any allergies and/or health challenges that we should be aware of?”; “What is your current hair state?”; “What is your hair type?”; “My hair texture feels:”; “I would describe the overall health of my hair as:”; What is your hair density (number of strands on your head)?”; “Please select your top 3 hair goals:”. In one or more embodiments, a personalized hair care plan module 315 may be configured to provide a variety of information, questions, or other types of prompts to initiate user input and user feedback similar to the GUI illustrated in FIG. 25, 2500.

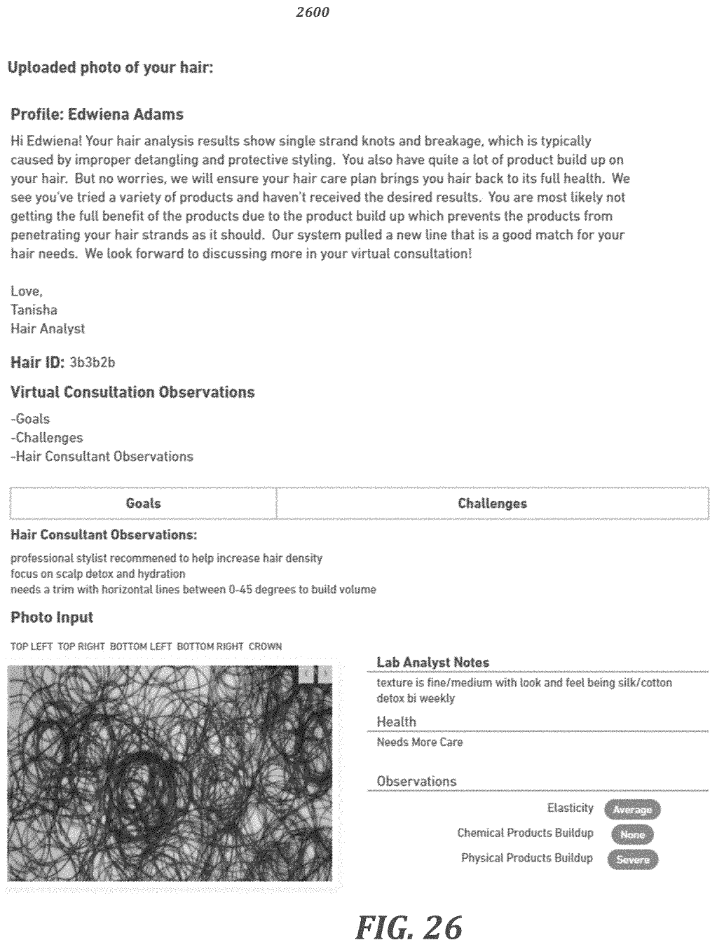

FIG. 26, 2600 shows a graphical user interface (GUIs) that may be presented by the hair care software application embodiment depicting a sample user profile. The user profile may include the following information but is not limited to: name, hair analysis results, uploaded photo of hair, message from a hair analyst, hair identification number (Hair ID), hair goals, hair challenges, hair consultant observations, lab analysts note, and other relevant information.

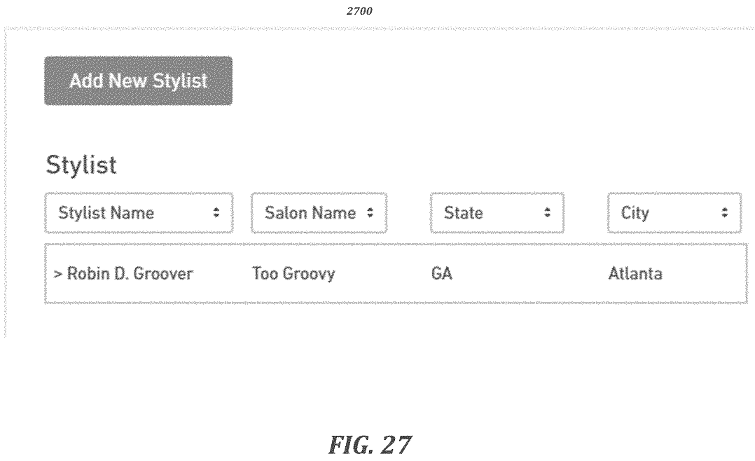

FIG. 27, 2700 shows a graphical user interface (GUIs) that may be presented by the hair care software application embodiment depicting the ability to add new stylists. The stylist information may include the following information but is not limited to: stylist name, salon name, state, city, description, and other relevant information.

The present disclosure provides a hair health assessment, diagnosis and recommendation platform. In one or more aspects, the hair health assessment, diagnosis and recommendation platform may be implemented and embodied as a hair health assessment, diagnosis and recommendation system. In one or more aspects, the system may further comprise a machine learning step wherein the machine learning step may be further configured to use artificial intelligence to return product matches and provide recommendations to one or more users profile based on the data analysis and classification of one or more hair specimens.

In one or more aspects, the system may further comprise a machine learning step wherein the machine learning step may be further configured to use artificial intelligence to provide guidance along one or more users' hair journey. The system may be further configured with the ability to take in user inputs to provide new recommendations as their hair and lifestyle changes based on the data analysis and classification of one or more hair specimens.

### Methods for Using the Devices and Systems

The present disclosure, according to further aspects, also provides methods of using the disclosed devices and systems. In one aspect, disclosed herein is a method for providing hair health assessment and recommendations.

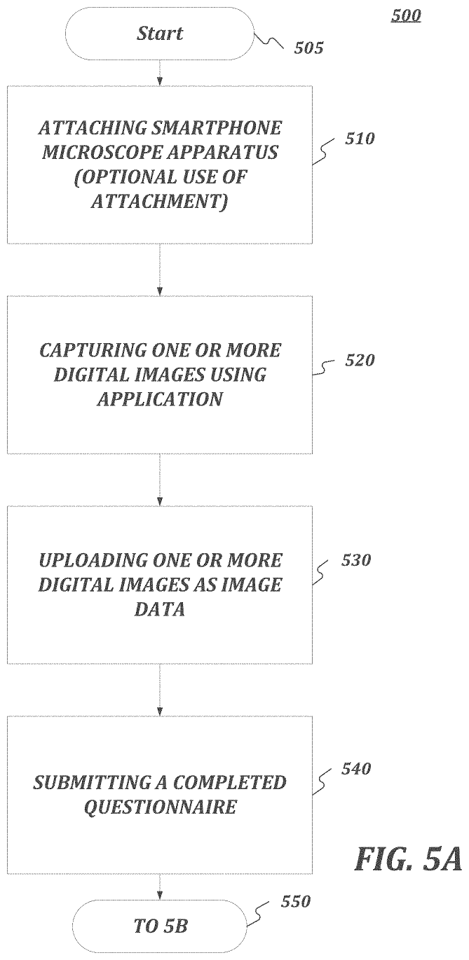

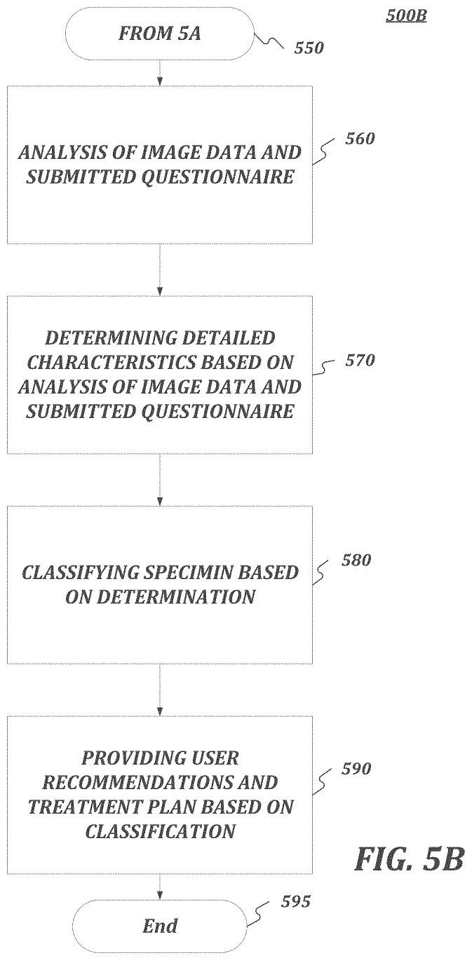

FIG. 5A and FIG. 5B show a flow chart of a method for providing Scientific and Technical Systems and Methods for Providing Hair Health Assessment and Styling Recommendations.

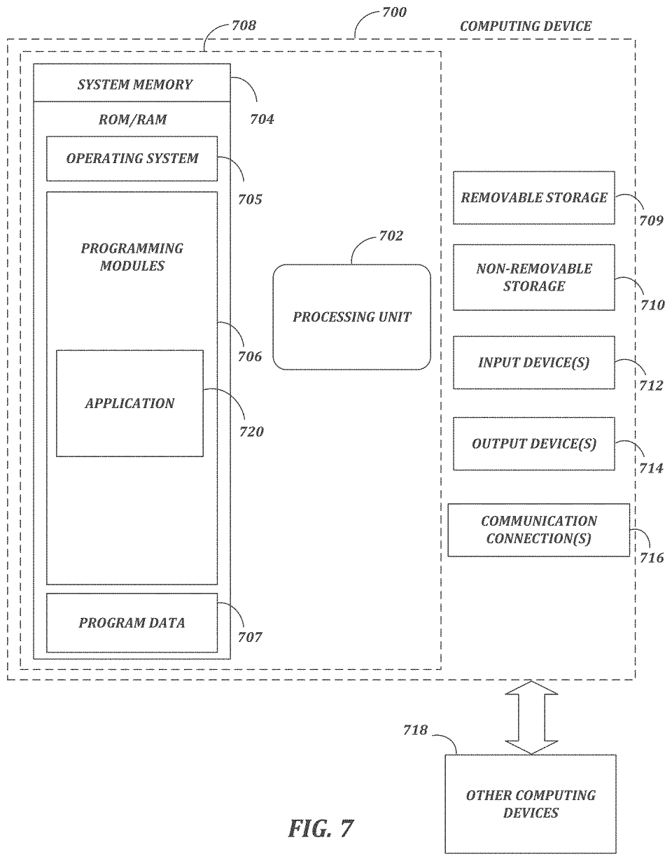

FIG. 7 is a block diagram of a system including a computing device for performing the various functions and methods disclosed herein.

### III. Platform Operation

FIG. 5A and FIG. 5B show a flow chart of a method for providing Scientific and Technical Systems and Methods for Providing Hair Health Assessment and Styling Recommendations.

FIG. 5A and FIG. 5B show a flow chart setting forth the general stages involved in a method 500 consistent with an embodiment of the disclosure for providing a hair assessment diagnosis and recommendation platform 100. Method 500 may be implemented using one of the computing devices 800a-800d (as shown in FIG. 1A, for example) as described in more detail below with respect to FIG. 5A and FIG. 5B.

In one or more aspects, the method may further comprise a machine learning step wherein the machine learning step may be further configured to use artificial intelligence to instantly return the product matches that may be most accurately paired with the profile attributes and hair data gathered from the hair strand images based on the data analysis and classification of one or more hair specimens.

All these steps collectively are pertinent for scalability and define our competitive advantage in the ability to service millions of users at any given time.

Although method 500 has been described to be performed by computing device 800, it should be understood that, in some embodiments, different operations may be performed by different networked elements in operative communication with computing device 800. For example, server 110 and/or computing device 800 may be employed in the performance of some or all of the stages in method 500. Moreover, server 110 may be configured much like computing device 800 and, in some instances, be one and the same embodiment. Similarly, apparatus 610 or 615 may be employed in the performance of some or all of the stages in method 500. Apparatus 610 or 615 may also be configured much like computing device 800.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 500 will be described in greater detail below.

Method 500 may begin at starting block 505 and proceed to stage 510 where a user 105 may attach a microscope apparatus (also referred to herein as the microscopic image capture device) to a mobile device. In some embodiments, stage 510 is an optional step. As described in detail above, an embedded digital camera of the user's mobile device may be used to capture images on a microscopic level, eliminating the need to attach a microscope, in some implementations For example, a user 105 may attach a microscopic attachment to a digital camera of a smartphone.

Method 500 may proceed to stage 520, where user 105 may capture one or more microscopic digital images. For example, a user 105 may use the microscopic image capturing device (situated as a front-facing camera of the mobile device) to capture an front image, angled image, or side profile image, and images on all sides of the head wherein the image comprises images from top left, top right, bottom left, bottom right, crown, and detailed microscopic images of one or more hair strands. Capturing the digital images can involve the user opening a mobile application installed on the mobile device (which is an aspect of the platform's software application) to open the functionality of the microscopic image capture device and take a picture of their hair at a microscopic-level of magnification.

Once user 105 captures one or more digital images in stage 520, method 500 may continue to stage 530. At stage 530 the user 105 may upload one or more image captures of the hair as a hair sample, or specimen to be subjected to the hair analysis features on the platform. Thus, the uploaded digital images may be analyzed as image data by the platform using AL/ML technology. For example, the user 105 may upload a front-facing image, angled image, or side profile image and an image on all sides of the head wherein the image comprises images from top left, top right, bottom left, bottom right, crown, and detailed microscopic images of one or more hair strands.

After the user 105 uploads one or more image captures as image data in stage 530, method 500 may proceed to stage 540 where user 105 may submit a completed questionnaire. It should be appreciated that stage 530 is an optional step in some embodiments. As an example, a questionnaire may be completed in order to provide additional information about the user that can ultimately be used to further personalize the recommendations for the particular user (based on the information submitted in the questionnaire). Restated, the process 500 can continue and successfully complete without stage 530. For example, user 105 may submit user data wherein user data comprises at least one of user feedback, a completed questionnaire, and user information. In some embodiments, the questionnaire is presented to the user for interaction on their respective computing device via the user-interface module. Once user 105 submits a completed questionnaire in stage 540, method 500 may then continue to 500B at stage 550.

Method 500B may begin at starting block 550 and proceed to stage 560 where computing device 800 or server 110 may analyze image data and submitted questionnaire data. Also, stage 560 can involve analyzing the image data, in instances when the questionnaire has optionally not been submitted. For example, computing device 800 or server 110 analyzing images including but not limited to images from the front, side, top left, top right, bottom left, bottom right, crown, and detailed images of one or more hair strands.

From stage 560, where computing device 800 or server 110 analyzes image data and submitted questionnaire, method 500B may advance to stage 570 where computing device 800 or server 110 may determine characteristics based on analysis. For example, computing device 800 or server 110 may identify one or more of: the texture, type, and condition of the hair strand by measuring the strand diameter, cuticle layer, hair shaft, ends, and other characteristics based on the analysis of image data and submitted user information. As a key aspect of the disclosed method 500, AI/ML is employed to determine the characteristics (or attributes) of the hair, such as texture, type, and condition of the hair strand by measuring the curl pattern, strand diameter, cuticle layer, hair shaft, ends, and detect other particles and observations. In addition, user input submitted through the questionnaire in previous stage 540 can be used to tag a profile corresponding to the user with the specific hair attributes (determined as a result of the hair analysis).

Once computing device 800 or server 110 determine characteristics based on analysis in stage 570, method 500B may continue to stage 580 where computing device 800 or server 110 may classify hair specimen information based on determination. For example, computing device 800 or server 110 may classify hair specimens including but not limited to detecting other particles and observations. Furthermore, stage 580 can determine a Hair ID that can be assigned to the hair of the user as a form of standard classification. Thus, the method 500 can utilize different Hair IDs to link a defined classification to the varying types of hair that may be seen by the platform.

After computing device 800 or server 110 classified hair specimen information based on determination in stage 580, method 500B may proceed to stage 590 where computing device 800 or server 110 may recommend a personalized hair care plan based on classification. For example, computing device 800 or server 110 may recommend a personalized hair care plan, user-based recommendations, scientific hair product recommendations, scientific ingredient recommendations, recommendation regarding overall health, as well as methods of assessment based on classification. As an example, an overall health summary can be provided as a recommendation, which also includes information on the current state of their hair. Again, as a key aspect of the disclosed method 500, and AI/ML technology is applied to instantly return recommendations and product matches that are most accurately paired with the profile attributes and hair data gathered from the hair strand images. In an embodiment, the hair care product matches and recommendations are returned in the profile corresponding to the particular user. Thus, the method 500 provides guidance along the user's hair and health journey with the ability to take in user inputs to provide new recommendations as the user's hair and lifestyle changes.

Once computing device 800 or server 110 recommends a personalized hair careplan based on classification in stage 590, method 500B may then end at stage 595.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications can be referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present disclosure is not entitled to antedate such publication by virtue of prior disclosure. Further, the dates of publication provided herein can be different from the actual publication dates, which can require independent confirmation.

The patentable scope of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

### Claims

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

Although very narrow claims are presented herein, it should be recognized the scope of this disclosure is much broader than presented by the claims. It is intended that broader claims will be submitted in an application that claims the benefit of priority from this application.

1. A computer-based system for hair health, assessment, styling diagnosis, and recommendation, the system comprising:
a computing device configured to transmit user-submitted data, comprising a digital image capture device configured to capture a microscopic digital image of a hair sample from a user;
a server, communicatively coupled to the computing device, comprising:
one or more processors;
a memory, and
machine-readable instructions stored on the memory and executed by the one or more processors to cause the server to:
apply machine learning (ML) and artificial intelligence (AI) to convert the microscopic digital image to hair data,
analyze the hair data and user-submitted data to determine a plurality of attributes associated with the hair sample, wherein each attribute comprises a code of alphanumeric characters,
determine a unique identifier associated with the user, wherein the unique identifier consists of an aggregate of a six-digit alphanumeric code for hair attributes, hair classification and user submitted data and
communicate one or more recommendations related to hair care based on the unique identifier.

2. (canceled)

3. The system of claim 1, wherein the microscopic digital image capture device comprises a microscopic lens attachment to a digital camera included on the computing device, or an embedded digital camera on the computing device.

4. (canceled)

5. The system of claim 1, wherein the system comprises a scanning electron microscope (SEM) capturing an additional microscopic digital image of the hair sample and communicating the additional microscopic digital image of the hair sample to the server as an additional hair sample.

6. The system of claim 5, wherein the microscopic digital image capture device is deployed at one or more locations comprising: a hair salon; a beauty supply store; and a retailer.

7. (canceled)

8. The system of claim 1, wherein the machine-readable instructions further cause the machine-readable instructions to measure the hair sample of the microscopic digital image to obtain two or more measurements comprising: a curl pattern of the hair sample, a strand diameter of the hair sample, the condition and physical attributes of the hair sample, an elasticity of the hair sample, and a porosity of the hair sample.

9. The system of claim 8, wherein the attributes associated with the hair sample are based on the measurements and comprise one or more of: type, texture, condition, porosity, elasticity, and density.

10. The system of claim 1, wherein the one or more recommendations related to hair care comprises one or more of: a recommendation of a hair care regimen, a recommendation of hair care products, a recommendation of a hair style, a recommendation of ingredients, a recommendation of hair techniques, and a recommendation of professional hair services.

11. A method comprising:
capturing one or more digital images of hair using a microscopic apparatus or an embedded digital camera of a mobile device;
uploading the one or more digital images as image data;
submitting user data, wherein the user data comprises at least one of user feedback, a completed questionnaire, and user information;
applying machine learning (ML) and artificial intelligence (AI) to convert the image data to hair data;
analyzing the hair data and user-submitted data to determine a plurality of attributes associated with the hair, wherein each attribute comprises a code of alphanumeric characters;
determining a unique identifier associated with the user, wherein the unique identifier comprises an array comprising each code of alphanumeric characters associated with each attribute and user submitted data;
classifying the hair based on determined unique identifier wherein the unique identifier is an aggregate of a six-digit alphanumeric code for hair attributes, hair classification and user data; and
recommending a personalized hair care plan based on classification.

12. The method of claim 11, wherein recommending a personalized hair care plan further comprises at least two or more of a hair assessment, a hair care product, a hair care regimen, and a hair styling plan.

13. (canceled)

14. The method of claim 11, wherein recommending the personalized hair care plan comprises, determining at least one hair care product that matches the attributes associated with the hair.

15. The method of claim 14, wherein determining a match comprises determining that one or more ingredients of the hair care product corresponds to one or more of the determined attributes associated with the hair.

16. A computer-readable medium storing instructions that, when executed by a computing device, cause the computing device to perform:
capturing one or more digital images of hair, wherein capturing the one or more digital images comprises activating an apparatus configured to capture a microscopic image of the hair;
uploading the one or more digital images as image data;
submitting user data, wherein the user data comprises at least one of: user feedback, a completed questionnaire, and user information;
applying machine learning (ML) and artificial intelligence (AI) to convert the one or more digital images to hair data;
analyzing the hair data and user-submitted data to determine a plurality of attributes associated with the hair, wherein each attribute comprises a corresponding plurality of codes of alphanumeric characters; and
determining a unique identifier associated with the user, wherein the unique identifier comprises an aggregate array comprising each corresponding plurality of codes of alphanumeric characters associated with each attribute and user-submitted data.

17. The computer-readable medium of claim 16, causing the computing device to further perform:
receiving a recommendation of a personalized hair care plan based on the unique identifier associated with the hair.

18-20. (canceled)
